(19) United States
(12) Patent Application Publication
(10) Pub. No.: US 2023/0055834 A1
(43) Pub. Date: Feb. 23, 2023

(54) LIQUID DRUG PUMPS INCLUDING USER FEEDBACK INDICATING PUMP ORIENTATION

(71) Applicant: JANSSEN BIOTECH, INC., Horsham, PA (US)

(72) Inventors: Jimmy Vinh Hoang Cassebee, Fremont, CA (US); Peter KRULEVITCH, Milpitas, CA (US); Michael CANNAMELA, Atlantic Highland, NJ (US); Brandon MCKENZIE, West Chester, PA (US); Steven M. VESOLE, San Bruno, CA (US)

(21) Appl. No.: 17/785,778

(22) PCT Filed: Dec. 4, 2020
(86) PCT No.: PCT/IB2020/061503

§ 371 Date: Jun. 15, 2022

Related U.S. Application Data
(60) Provisional application No. 62/950,309, filed on Dec. 19, 2019.

(51) Int. Cl. A61M 5/172; A61M 5/142; A61M 5/145

(52) U.S. Cl. CPC A61M 5/172; A61M 5/14244; A61M 5/145; A61M 2205/502; A61M 2205/3331; A61M 2205/581; A61M 2205/582; A61M 2205/583

(57) ABSTRACT

Various exemplary liquid drug pumps including user feedback indicating pump orientation are provided. In general, a pump configured to deliver a liquid drug to a patient includes a user interface configured to indicate an orientation of the pump. The pump also includes a reservoir configured to contain the drug therein, a conduit configured to receive the drug therein from the reservoir, a needle or cannula in fluid communication with the conduit and configured to deliver the drug therethrough to a patient wearing the pump, a fluid path fluidly connecting the reservoir and the needle or cannula, and a pumping assembly configured to pump the fluid through the fluid path from the reservoir to the needle or cannula. The user interface indicating the pump's orientation allows a user of the pump to know whether or not the pump is in a desired orientation for delivery of the drug to the patient.

### FIELD

The present disclosure relates generally to liquid drug pumps including user feedback indicating pump orientation.

### BACKGROUND

Pharmaceutical products (including large and small molecule pharmaceuticals, hereinafter “drugs”) are administered to patients in a variety of different ways for the treatment of specific medical indications. A pump is a type of drug administration device that can administer a liquid drug to the patient. Some pumps are wearable by a patient and can include a reservoir, such as a vial or a cartridge, that contains the liquid drug therein for delivery to the patient through a needle inserted into the patient.

The drug can be removed from the reservoir through a conduit and delivered to the patient through the needle. However, if the conduit is not in complete communication with the liquid drug in the reservoir, air can enter the conduit with the drug or instead of the drug. Air is undesirable to deliver to the patient because of, e.g., patient discomfort. If the conduit is not in complete communication with the liquid drug in the reservoir, the patient's desired treatment is interrupted by the pump delivering only air to the patient instead of the drug, by the pump delivering air to the patient with only a partial intended dose of the drug, or by the pump not delivering any air or any drug to the patient due to a detected error of air entering the conduit from the reservoir. Interrupting the patient's treatment may adversely affect the patient's health and may cause patient frustration with the pump and thereby reduce the patient's willingness to use the pump in the future as recommended by the patient's health care provider.

The conduit may not be in complete communication with the liquid drug in the reservoir for a variety of reasons. For example, the conduit may not be in complete communication with the liquid drug in the reservoir due to the patient's orientation when the drug is being pumped out of the reservoir and into the patient via the needle. Liquid in the reservoir naturally settles at a location therein due to gravity, so depending on the patient's orientation, the liquid drug may not settle within the reservoir at a location where the conduit is in complete communication with the liquid drug. Additionally, for pumps that deliver multiple doses of the drug over time, it becomes more likely over time that the patient's orientation will adversely affect the conduit's accessibility of the drug within the reservoir. As the amount of the drug in the reservoir decreases, there is less drug present in the reservoir to be in complete communication with the conduit.

For another example, the conduit may not be in complete communication with the liquid drug in the reservoir due to the pump not being positioned properly on the patient. The pump will typically come with instructions indicating how the pump should be attached to the patient, including a recommended orientation of the pump relative to the patient. The recommended orientation may help maximize the conduit's ability to be in complete communication with the drug in the reservoir for every delivery of the drug to the patient. However, the pump may not be attached to the patient at the recommended orientation due to unintentional user error.

Accordingly, there remains a need for pumps with improved liquid drug accessibility.

### SUMMARY

In general, liquid drug pumps including user feedback indicating pump orientation are provided.

In one aspect, a pump configured to deliver a liquid drug to a patient is provided that in one embodiment includes a reservoir configured to contain the liquid drug therein, a pumping assembly configured to drive the liquid drug from the reservoir for delivery to the patient, a sensor configured to measure an orientation of the pump, a user interface, and control circuitry configured to receive data from the sensor indicative of the measured orientation of the pump and to cause the user interface to provide an indication of the orientation of the pump to a user of the pump. The pump can vary in any number of ways.

In another embodiment, a pump configured to deliver a liquid drug to a patient includes a housing. The housing includes a first side configured to be attached to skin of a patient at a recommended orientation relative to the patient. The pump also includes a reservoir in the housing. The reservoir is configured to contain the liquid drug therein. The pump also includes a pumping assembly in the housing. The pumping assembly is configured to drive the liquid drug from the reservoir for delivery to the patient. The pump also includes a sensor in the housing. The sensor is configured to measure an orientation of the pump. The pump also includes a user interface located on a second side of the housing. The user interface is configured to be visible by the patient with the first side of the housing attached to the skin of the patient. The pump can vary in any number of ways.

In another aspect, a method of using a pump configured to deliver a liquid drug to a patient is provided and in one embodiment includes control circuitry of the pump receiving data from a sensor of the pump as a series of real time pump orientation measurements, and the control circuitry causing a user interface of the pump to provide an indication of the orientation of the pump as a series of real time indications that each correspond to one of the real time pump orientation measurements. The method can have any number of variations.

### BRIEF DESCRIPTION OF DRAWINGS

The present invention is described by way of reference to the accompanying figures which are as follows:

FIG. 1 is a schematic view of an embodiment of a pump configured to deliver a liquid drug to a patient;

FIG. 2 is a perspective view of an area of accessibility for a conduit of the pump of FIG. 1,

FIG. 3 is a side view of an embodiment of a reservoir of the pump of FIG. 1 in various orientations;

FIG. 4 is a perspective view of an embodiment of the pump of FIG. 1 including a housing;

FIG. 5 is a perspective view of an embodiment of the pump of FIG. 1 including a housing and a housing mount releasably attached to the housing;

FIG. 6 is a perspective view of the housing and housing mount of FIG. 5 with the housing removed from the housing mount;

FIG. 7 is a perspective view of the housing and housing mount of FIG. 6 with the housing reattached to the housing mount;

FIG. 8 is a schematic view of another embodiment of a pump configured to deliver a liquid drug to a patient and an embodiment of a reservoir configured to be received in the pump;

FIG. 9 is a schematic view of the reservoir and pump of FIG. 8 coupled together;

FIG. 10 is a schematic view of the reservoir and pump of FIG. 9 with a conduit of the pump penetrated into the reservoir;

FIG. 11 is a schematic view of yet another embodiment of a pump configured to deliver a liquid drug to a patient;

FIG. 12 is a perspective view of another embodiment of a pump configured to deliver a liquid drug to a patient;

FIG. 13 is a perspective view of the pump of FIG. 12 with a first light of the pump illuminated;

FIG. 14 is a perspective view of the pump of FIG. 13 with the first light and a second light of the pump illuminated;

FIG. 15 is a perspective view of the pump of FIG. 14 with one of a series of lights of the second light unilluminated and a remainder of the series of lights of the second light illuminated;

FIG. 16 is a perspective view of the pump of FIG. 15 with half of the series of lights of the second light unilluminated and the other half of the series of lights of the second light illuminated;

FIG. 17 is a perspective view of the pump of FIG. 16 with all but one of the series of lights of the second light unilluminated;

FIG. 18 is a perspective view of the pump of FIG. 17 with the second light unilluminated and the first light illuminated;

FIG. 19 is a perspective view of the pump of FIG. 15 with a second one of the series of lights of the second light illuminated in a different color;

FIG. 20 is a perspective view of the pump of FIG. 13 with the first light and a second light of the pump illuminated in a different color;

FIG. 21 is a perspective view of yet another embodiment of a pump configured to deliver a liquid drug to a patient;

FIG. 22 is a perspective view of the pump of FIG. 21 with a light of the pump illuminated;

FIG. 23 is a perspective view of the pump of FIG. 21 with a first one of a series of lights of the light illuminated and a remainder of the series of lights of the light unilluminated;

FIG. 24 is a perspective view of the pump of FIG. 23 with a second one of the series of lights of the light illuminated and a remainder of the series of lights of the light unilluminated;

FIG. 25 is a perspective view of the pump of FIG. 24 with a third one of the series of lights of the light illuminated and a remainder of the series of lights of the light unilluminated;

FIG. 26 is a perspective view of the pump of FIG. 25 with a fourth one of the series of lights of the light illuminated and a remainder of the series of lights of the light unilluminated;

FIG. 27 is a perspective view of the pump of FIG. 26 with the series of lights of the light illuminated;

FIG. 28 is a perspective view of the pump of FIG. 27 with the first one of the series of lights of the light unilluminated and a remainder of the series of lights of the light illuminated;

FIG. 29 is a perspective view of the pump of FIG. 28 with the first and second ones of the series of lights of the light unilluminated and a remainder of the series of lights of the light illuminated;

FIG. 30 is a perspective view of the pump of FIG. 29 with the first, second, and third ones of the series of lights of the light unilluminated and a remainder of the series of lights of the light illuminated;

FIG. 31 is a perspective view of the pump of FIG. 30 with the series of lights of the light illuminated;

FIG. 32 is a perspective view of the pump of FIG. 28 with the first one of the series of lights of the light unilluminated, the second one of the series of lights of the light illuminated in a second color, and a remainder of the series of lights of the light illuminated in a first color;

FIG. 33 is a perspective view of the pump of FIG. 21 with the series of lights of the light illuminated in the second color;

FIG. 34 is a perspective view of still another embodiment of a pump configured to deliver a liquid drug to a patient;

FIG. 35 is a perspective view of the pump of FIG. 34 with a first light of the pump illuminated;

FIG. 36 is a perspective view of the pump of FIG. 35 with the first light and a second light of the pump illuminated;

FIG. 37 is a perspective view of the pump of FIG. 36 with one of a series of lights of the second light unilluminated and a remainder of the series of lights of the second light illuminated;

FIG. 38 is a perspective view of the pump of FIG. 37 with half of the series of lights of the second light unilluminated and the other half of the series of lights of the second light illuminated;

FIG. 39 is a perspective view of the pump of FIG. 38 with all but one of the series of lights of the second light unilluminated;

FIG. 40 is a perspective view of the pump of FIG. 39 with the first and second lights unilluminated;

FIG. 41 is a perspective view of the pump of FIG. 37 with a second one of the series of lights of the second light illuminated in a different color;

FIG. 42 is a perspective view of the pump of FIG. 34 with the first light and a second light of the pump illuminated in a different color;

FIG. 43 is a perspective view of yet another embodiment of a pump configured to deliver a liquid drug to a patient;

FIG. 44 is a perspective view of the pump of FIG. 43 with a sticker having been removed therefrom;

FIG. 45 is a perspective view of another embodiment of a pump configured to deliver a liquid drug to a patient;

FIG. 46 is another perspective view of the pump of FIG. 45;

FIG. 47 is a perspective view of still another embodiment of a pump configured to deliver a liquid drug to a patient;

FIG. 48 is another perspective view of the pump of FIG. 47;

FIG. 49 is a perspective view of yet another embodiment of a pump configured to deliver a liquid drug to a patient;

FIG. 50 is another perspective view of the pump of FIG. 49; and

FIG. 51 is a schematic view of an embodiment of a system including a pump configured to deliver a liquid drug to a patient and an external device configured to communicate with the pump.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary liquid drug pumps including user feedback indicating pump orientation are provided. In general, a pump configured to deliver a liquid drug to a patient includes a user interface configured to indicate an orientation of the pump. The pump also includes a reservoir configured to contain the liquid drug therein, a conduit configured to receive the drug therein from the reservoir, a needle or cannula in fluid communication with the conduit and configured to deliver the drug therethrough to a patient wearing the pump, a fluid path fluidly connecting the reservoir and the needle or cannula, and a pumping assembly configured to pump the fluid through the fluid path from the reservoir to the needle or cannula. The user interface indicating the pump's orientation allows a user (e.g., the patient, the patient's care giver, the patient's doctor, etc.) of the pump to know whether or not the pump is in a desired orientation for delivery of the drug to the patient. The user may therefore be informed as to whether the patient's body and/or the pump should be reoriented until the pump is in a desired orientation for delivery of the drug to the patient. The desired orientation corresponds to an orientation of the pump relative to gravity, e.g., the ground, at which the liquid drug can be pumped out of the reservoir and into the conduit without any air entering the conduit from the reservoir. The user interface is thus configured to help ensure that the user begins the drug delivery process, e.g., by pressing a start button on the pump or otherwise triggering drug delivery, when the pump is oriented such that the conduit receives therein only drug from the reservoir for delivery to the patient and that the conduit does not receive therein any air contained in the reservoir. The patient may thereby be ensured to receive only drug through the needle or cannula and not any air through the needle or cannula, and the patient's drug dose(s) can therefore be fully delivered at a desired schedule without interruption since drug, and not any air, will be provided to the needle or cannula via the conduit.

The drug to be delivered using a pump as described herein can be any of a variety of drugs. Examples of drugs that can be delivered using a pump as described herein include antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, antiallergics, antihistamines, anti-inflammatories, corticosteroids, disease modifying anti-rheumatic drugs, erythropoietin, and vaccines.

The user feedback described herein can be used with a variety of drug delivery pumps configured to deliver a drug to a patient. Examples of drug delivery pumps include the pumps described in Intl. Pat. Pub. WO 2018/096534 entitled “Apparatus For Delivering A Therapeutic Substance” published May 31, 2018, in U.S. Pat. Pub. No. 2019/0134295 entitled “Local Disinfection For Prefilled Drug Delivery System” published May 9, 2019, in U.S. Pat. No. 7,976,505 entitled “Disposable Infusion Device Negative Pressure Filling Apparatus And Method” issued Jul. 12, 2011, and in U.S. Pat. No. 7,815,609 entitled “Disposable Infusion Device Positive Pressure Filling Apparatus And Method” issued Oct. 19, 2010, which are hereby incorporated by reference in their entireties. Other examples of drug delivery pumps include the SmartDose® Drug Delivery Platform available from West Pharmaceutical Services, Inc. of Exton, Pa., the OMNIPOD® available from Insulet Corp. of Acton, Mass., the YpsoDose® patch injector available from Ypsomed AG of Burgdorf, Switzerland, the BD Libertas™ wearable injector available from Becton, Dickinson and Co. of Franklin Lakes, N.J., the Sorrel Medical pump available from Sorrel Medical of Netanya, Israel, the SteadyMed PatchPump® available from SteadyMed Ltd. of Rehovot, Israel, the Sensile Medical infusion pump available from Sensile Medical AG of Olten, Switzerland, the SonceBoz wearable injectors available from SonceBoz SA of Sonceboz-Sombeval, Switzerland, enFuse® available from Enable Injections of Cincinnati, Ohio, the on-body injector for Neulasta® available from Amgen, Inc. of Thousand Oaks, Calif., the Pushtronex® System available from Amgen, Inc. of Thousand Oaks, Calif., and the Imperium® pump available from Unilife Corp. of King of Prussia, Pa.

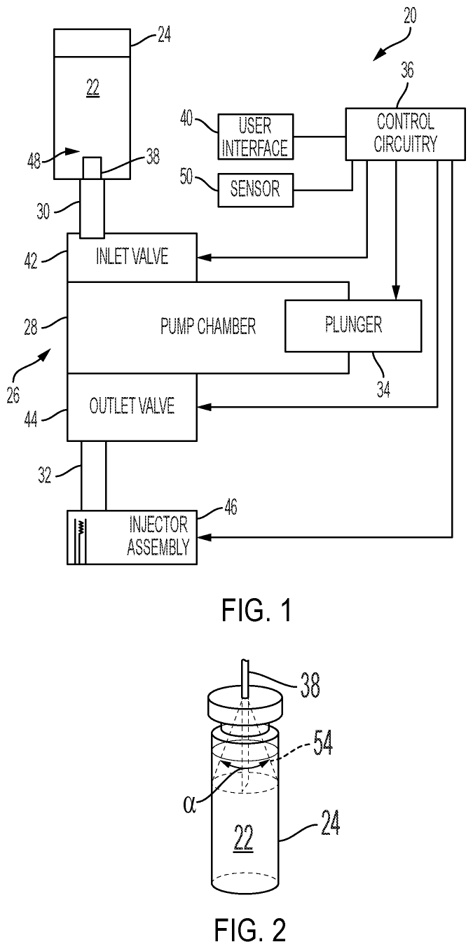

FIG. 1 illustrates an embodiment of a pump 20, e.g., a patch pump, configured to be worn by a patient and to deliver a drug (also referred to herein as a “therapeutic substance”) 22 to the patient. The pump 20 can be configured to be attached to the patient in any of a variety of ways, as will be appreciated by a person skilled in the art, such as by including a backing or label configured to be removed from a body of the pump 20 to expose adhesive attachable to the patient. The pump 20 includes a therapeutic substance reservoir 24 containing the drug 22 therein. The reservoir 24 can be prefilled by a medical vendor or device manufacturer, or the reservoir 24 can be filled by a user (e.g., the patient, the patient's caregiver, a doctor or other health care professional, a pharmacist, etc.) prior to use of the pump 20. Alternatively, the reservoir 24 can come prefilled from a medical vendor ready to be loaded or inserted into pump 20 prior to use. The pump 20 also includes a conduit 38 through which the drug 22 is configured to pass from the reservoir 24 and into an inlet fluid path 30 operatively connected to an injector assembly 46 of the pump 20 that is configured to deliver the therapeutic substance 22 into a patient. The conduit 38 is thus a tube in which the drug 22 can flow.

The pump 20 also includes a user interface 40 configured to indicate an orientation of the pump 20 to a user of the pump 20, e.g., the patient wearing the pump 20 by having the pump removably attached thereto using adhesive or other attachment mechanism, the patient's care giver assisting the patient in using the pump 20, a health care professional assisting the patient in using the pump 20, etc. The user interface 40 can have a variety of configurations, as discussed further below. The user interface 40 indicating the orientation of the pump 20 helps ensure that the conduit 38 is in complete communication with the drug 22 in the reservoir 24 at least when the conduit 38 is receiving the drug 22 therein from the reservoir 24, e.g., under force of an electromechanical pumping assembly 26 of the pump 20, regardless of an orientation of the patient wearing the pump 20, e.g., regardless of whether the patient is standing, sitting, lying down, bent over, etc. The user interface 40 may therefore be configured to help ensure that the drug 22, but not air, enters the conduit 38 from the reservoir 24. FIG. 1 shows the conduit 38 in complete communication with the drug 22 in the reservoir 24.

The pump 20 also includes a sensor 50 configured to monitor an orientation of the pump 20 relative to gravity, e.g., the ground. Examples of the sensor 50 configured to monitor orientation include an accelerometer, an inertial measurement unit (IMU), and a MARG (magnetic, angular rate, and gravity) sensor. In an exemplary embodiment the sensor 50 is a single sensor, which may help reduce cost of the pump 20, help conserve space within the pump 20 for other components, and/or help reduce an overall size of the pump 20. The sensor 50 can, however, include a plurality of sensors, which may help provide redundancy and allow for orientation measurements to be confirmed with one another for accuracy.

The electromechanical pumping assembly 26, e.g., a motor thereof, is operatively connected to the reservoir 24 and is configured to cause delivery of the therapeutic substance 22 to the patient via the injector assembly 46, e.g., through a needle or cannula of the injector assembly 46 that has been inserted into the patient. The electromechanical pumping assembly 26 is shaped to define a rigid pump chamber 28 that includes a therapeutic substance inlet 30 through which the therapeutic substance 22 is received from the conduit 30, and hence from the reservoir 24, into the pump chamber 28. The rigid pump chamber 28 also includes a fluid path outlet 32 through which the therapeutic substance 22 is delivered from the pump chamber 28 to the patient via the injector assembly 46. Although the pumping assembly 26 is electromechanical in this illustrated embodiment, the pumping assembly of the pump 20 (and for other embodiments of pumps described herein) can instead be mechanical. The mechanical pumping assembly need not include any electronic components or controls. For example, the mechanical pumping assembly can include a balloon diaphragm configured to be activated to cause delivery of a drug through mechanical action.

The pump 20 also includes a plunger 34 slidably disposed within the pump chamber 28 and sealably contacting an inside of the pump chamber 28. The plunger 34 is configured to be in direct contact with the drug 22 in the pumping chamber 28.

The pump 20 also includes control circuitry 36. The control circuitry 36 is operatively connected to the sensor 50 and is configured to receive measurement data from the sensor 50, e.g., to receive a signal from the sensor 50 indicative of a sensed orientation. The control circuitry 36 is also operatively connected to the user interface 40 and is configured to cause the user interface 40 to provide information to the user indicating the orientation of the pump 20 based on the pump's orientation as measured by the sensor 50. The control circuitry 36 is configured to receive data from the sensor 50 in real time with the sensor's sensing and to cause pump orientation information to be provided via the user interface 40 in real time such that the user interface 40 indicates a current orientation state of the pump 20.

The electromechanical pumping assembly 26 is configured to be driven to operate in two pumping phases by the control circuitry 36. In a first pumping phase, the control circuitry 36 is configured to drive the plunger 34 (e.g., slidably move the plunger 34 in the pump chamber 28) to draw the drug 22 from the reservoir 24 into the conduit 38, then into the inlet fluid path 30, and then through an inlet valve 42 and into the pump chamber 28. The inlet valve 42 is configured to be opened and closed such that when the inlet valve 42 is open there is fluid communication between the reservoir 24 and the pump chamber 28, and when the inlet valve 42 is closed there is no fluid communication between the reservoir 24 and the pump chamber 28. During the first pumping phase, the control circuitry 36 is configured to cause the inlet valve 42 to open, cause an outlet valve 44 to close, and drive the plunger 34 to draw the therapeutic substance 22 from the reservoir 24 into the pump chamber 28, e.g., the control circuitry 36 is configured to set the inlet valve 42 and the outlet valve 44 such that the therapeutic substance 22 can flow only between the reservoir 24 and the pump chamber 28. Thus, as the plunger 34 is drawn back, the therapeutic substance 22 is drawn into pump chamber 28. The control circuitry 36 causing the inlet valve 42 to open and the outlet valve 44 to close can be active control or can be passive control in which the valves 42, 44 are mechanical valves that automatically open/close due to the driving of the plunger 34.

In a second pumping phase, the control circuitry 36 is configured to drive the plunger 34 to deliver the drug 22 from the pump chamber 28 through the outlet valve 44 to the outlet fluid path 32 and then to the injector assembly 46 for delivery into the patient. The outlet valve 44 is configured to be opened and closed such that when the outlet valve 44 is open there is fluid communication between the pump chamber 28 and the patient, and when the outlet valve 44 is closed there is no fluid communication between the pump chamber 28 and the patient. During the second pumping phase, the control circuitry 36 is configured to cause the inlet valve 42 to close, cause the outlet valve 44 to open, and drive the plunger 34 to deliver the therapeutic substance 22 from the pump chamber 28 in a plurality of discrete motions of the plunger 34. For example, the control circuitry 36 can be configured to set the inlet valve 42 and the outlet valve 44 such that the therapeutic substance 22 can flow only between the pump chamber 28 and the patient, and the plunger 34 is incrementally pushed back into the pump chamber 28 in a plurality of discrete motions thereby delivering the therapeutic substance 22 to the patient in a plurality of discrete dosages. Similar to that discussed above, the control circuitry 36 causing the inlet valve 42 to close and the outlet valve 44 to open can be active control or can be passive control in which the valves 42, 44 are mechanical valves that automatically open/close due to the driving of the plunger 34.

In some embodiments, the control circuitry 36 is configured to drive the plunger 34 to draw the therapeutic substance 22 into the pump chamber 28 in a single motion of the plunger 34, e.g., the plunger 34 is pulled back in a single motion to draw a volume of the therapeutic substance 22 into the pump chamber 28 during the first pumping phase. Alternatively, the control circuitry 36 can be configured to drive the plunger 34 to draw the therapeutic substance 22 into the pump chamber 28 in one or more discrete expansion motions of the plunger 34, e.g., the plunger 34 can be pulled halfway out of the pump chamber 28 in one motion and then the rest of the way out of the pump chamber 28 in a second, separate motion. In this case, a duration of some or all expansion motions of the plunger 34 during the first pumping phase are typically longer than a duration of any one of the plurality of discrete motions of the plunger 34 during the second pumping phase.

In other embodiments, the control circuitry 36 is configured to drive the plunger 34 such that a duration of the first pumping phase and a duration of the second pumping phase are unequal. For example, a duration of the second pumping phase can be in a range of five to fifty times longer than the first pumping phase, e.g., at least ten times, thirty times, fifty times, etc. longer than a duration of the first pumping phase.

The pump 20 can also include a power supply (not shown) configured to provide power to components requiring power to operate, such as the control circuitry 36 and the sensor 50. In an exemplary embodiment, the power supply is a single power supply configured to provide power to each component of the pump 20 requiring power to operate, which may help reduce cost of the pump 20, help conserve space within the pump 20 for other components, and/or help reduce an overall size of the pump 20. The power supply can, however, include a plurality of power supplies, which may help provide redundancy and/or help reduce cost of the pump 20 since some components, e.g., the control circuitry 36 and/or the sensor 50, may be manufactured with an on-board dedicated power supply. In an exemplary embodiment, the power supply is on-board the pump 20, which may facilitate use of the pump 20 at any time in any location. In other embodiments, the power supply can include a mechanism configured to connect the pump 20 to an external power supply.

The control circuitry 36 is configured to determine whether the pump 20 is at an orientation, as indicated by the pump's current orientation as measured by the sensor 50, within a predefined range of predetermined acceptable orientations. The range of predetermined acceptable orientations is defined by an area of accessibility for the conduit 38 being in complete communication with the drug 22 in the reservoir 24. The range of predetermined acceptable orientations is stored in a memory of the control circuitry 36 for operative access by a processor of the control circuitry 36.

FIG. 2 illustrates an area 54 of accessibility for the conduit 38 being in complete communication with the drug 22 in the reservoir 24. The reservoir 24 in an exemplary embodiment and as shown in FIG. 2 is a vial, but the reservoir 24 can have other forms, as will be appreciated by a person skilled in the art, such as a cartridge. The area 54 has a cone shape, in particular a right circular cone shape, with the conduit 38 extending along a central axis of the cone along a height of the cone. An angle α of the cone's apex to a point along the cone's circular base perimeter is about 30°. With the reservoir 24 oriented anywhere within the area 54 of accessibility, the conduit 38 can access about 99% of the drug 22 contained in the reservoir 24 with the pump 20 in the predefined range of predetermined acceptable orientations. A person skilled in the art will appreciate that a value may not be precisely equal to a value but nevertheless be considered to be about that value due to any number of factors, such as manufacturing tolerance and sensitivity of measurement equipment.

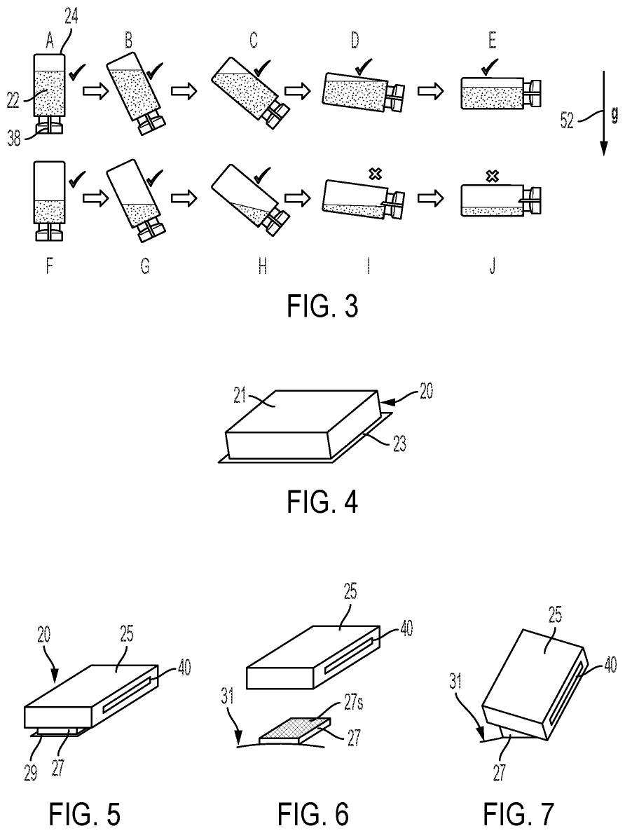

FIG. 3 illustrates ten possible relative positions A-J of the drug 22 and the conduit 38 in the reservoir 24. The reservoir 24 in an exemplary embodiment and as shown in FIG. 3 is a vial, but the reservoir 24 can have other forms, as will be appreciated by a person skilled in the art, such as a cartridge. A direction of gravity g is shown by arrow 52. Position A corresponds to the position of the drug 22 and the conduit 38 in the reservoir 24 with the pump 20 attached to the patient in accordance with the pump's provided instructions and with the patient standing or sitting upright, e.g., with the patient vertical. Position A is shown in FIG. 1. Position J corresponds to the position of the drug 22 and the conduit 38 in the reservoir 24 with the pump 20 attached to the patient in accordance with the pump's provided instructions and with the patient lying down, e.g., with the patient horizontal. Positions A-J are sequential positions as the patient moves from standing or sitting upright to lying down. Additional relative positions of the drug 22 and the conduit 38 in the reservoir 24 are possible between each of the illustrated ten positions A-J, and relative positions are also possible about other axes than the one illustrated in FIG. 3, but are not shown for ease of illustration and discussion. In each of positions A-H, the conduit 38 is in complete communication with the drug 22, as indicated by a check mark next to each of positions A-H. In each of positions I and J, the conduit 38 is not in complete communication with the drug 22, as indicated by an “x” next to each of positions I and J. The conduit 38 is not in complete communication with the drug 22 in positions I and J due to the pump's orientation relative to gravity g with the liquid drug 22 having settled in the reservoir 24 in response to gravity g.

Referring again to FIG. 1, the pump 20 can include a grip feature configured to communicate a preferred manual handling of the pump 20 to encourage attachment of the pump 20 to the patient at a recommended orientation relative to the patient. The grip feature encourages the patient to hold the pump 20 in a certain way by hand and thereby encourages the pump 20 to be held at an orientation at which the pump 20 can be effectively adhered to the patient at the recommended orientation. The grip feature is therefore configured to cooperate with the user interface 40 to encourage proper orientation of the pump 20 for drug delivery. Examples of the grip feature include one or more finger rests, a thumb rest, and a palm rest. The pump 20 can include any number of grip features, e.g., all of finger rest(s), a thumb rest, and a palm rest, two of finger rest(s), a thumb rest, and a palm rest, or only one of finger rest(s), a thumb rest, and a palm rest.

The pump 20 can include branding, e.g., a brand name of the pump 20 and/or a brand name of the drug 22, on an outer surface of a housing of the pump 20. The branding can be provided on the body in any of a variety of ways, such as by being printed on the housing, being on a sticker adhered to the housing, being etched into the housing, etc. In an exemplary embodiment, the branding is located on a side of the housing that is visible to and readable right-side-up by the patient when the pump 20 is attached to the patient at a recommended orientation relative to the patient. The branding thereby encourages attachment of the pump 20 to the patient at the recommended orientation relative to the patient since a user will tend to attach the pump 20 with the branding being readable right-side-up by the patient. The branding is therefore configured to cooperate with the user interface 40 (and the grip feature, if present) to encourage proper orientation of the pump 20 for drug delivery.

The user interface 40 can have a variety of configurations. In an exemplary embodiment, the user interface 40 includes a light, e.g., a light emitting diode (LED) or other type of light, configured to illuminate to provide an indication of the pump's orientation.

In some embodiments, the light is a single light. The single light can be located on the pump 20 at a location where the light would be visible to the patient wearing the pump 20 when the pump 20 is attached to the patient in accordance with the pump's instructions indicating how the pump should be attached to the patient, including a recommended orientation of the pump 20 relative to the patient. The single light can be configured to be illuminated, as controlled by the control circuitry 36, when the pump 20 is determined to be within the predefined range of predetermined acceptable orientations, and to not be illuminated, as controlled by the control circuitry 36, when the pump 20 is determined to not be within the predefined range of predetermined acceptable orientations. The user can therefore be informed whether or not the pump 20 is in an acceptable orientation for drug delivery by the light either being on (indicating an acceptable orientation for drug delivery) or off (indicating an unacceptable orientation for drug delivery). The user may therefore know to reorient the patient's body, and hence the orientation of the pump 20, until the light is illuminated. Alternatively, the single light can be configured to be a first color, as controlled by the control circuitry 36, when the pump 20 is determined to be within the predefined range of predetermined acceptable orientations, and to be a second, different color, as controlled by the control circuitry 36, when the pump 20 is determined to not be within the predefined range of predetermined acceptable orientations. The user can therefore be informed whether or not the pump 20 is in an acceptable orientation for drug delivery by the light either being the first color (indicating an acceptable orientation for drug delivery) or the second color (indicating an unacceptable orientation for drug delivery). In an exemplary embodiment, green can be the first color as a typically identifiable “start” or “go” color, and the second color can be red as a typically identifiable “stop” color.

In other embodiments, the light is a plurality of lights. In such embodiments, the plurality of lights can be configured similar to the single light either by all being illuminated or all being in a first color to indicate the pump 20 being within the predefined range of predetermined acceptable orientations or by all not being illuminated or all being in a second color to indicate the pump 20 not being within the predefined range of predetermined acceptable orientations. Alternatively, the plurality of lights can be configured as an electronic level with the plurality of lights arranged in a line, such as by using an LED strip. A center one (or ones) of the line of lights can be configured to be illuminated with a remainder of the lights not being illuminated (or illuminated in a different color than the center one(s)), as controlled by the control circuitry 36, when the pump 20 is determined to be within the predefined range of predetermined acceptable orientations, and for left or right ones of the lights to be illuminated, as controlled by the control circuitry 36, when the pump 20 is determined to not be within the predefined range of predetermined acceptable orientations either by being oriented too far left (left light(s) illuminated) or too far right (right light(s) illuminated). The electronic level can thus be configured to inform the user of whether or not the pump 20 is in an acceptable orientation for drug delivery in addition to indicating which direction the pump 20 should be moved in order to move the pump 20 from an unacceptable orientation for drug delivery to an acceptable orientation for drug delivery. The electronic level defined by a plurality of lights can have configuration other than a line of lights, such as by being a bullseye level including concentric rings, with illumination of a center-most one of the rings (or a central circle within concentric rings) being indicative of the pump 20 being determined to be within the predefined range of predetermined acceptable orientations with each one of the rings successively closer to the center ring (or center circle) being indicative of the pump 20 being determined to be closer to the predefined range of predetermined acceptable orientations.

The plurality of lights can be located on the pump 20 at a location where the lights would be visible to the patient wearing the pump 20 when the pump 20 is attached to the patient in accordance with the pump's instructions indicating how the pump should be attached to the patient, including a recommended orientation of the pump 20 relative to the patient. Providing a plurality of lights on the pump 20 instead of a single light allows the lights to be located on the pump 20 such that one or more of the lights are positioned to be visible by the patient wearing the pump 20 regardless of an angle at which the patient is looking at the pump 20, such as by the lights being positioned around a perimeter of the pump 20. The angle at which the patient is looking at the pump 20 can be different based on the patient's position, e.g., the patient lying on their side versus the patient standing up, the patient's head being tilted left versus being tilted right, etc. Also, if the pump 20 is not attached to the patient at the recommended orientation relative to the patient such that the patient's vantage point of the pump 20 is not the expected vantage point, at least one of the plurality of lights can still be visible to the patient.

Whether a single light or a plurality of lights, the light(s) can be arranged around a perimeter of a “start” button of the pump 20 that is configured to be depressed by a user to begin drug delivery from the pump 20. The user may therefore be less likely to not see the light(s) before beginning drug delivery since the light(s) are located next to the “start” button that the user will tend to look at when pushing the “start” button.

Whether a single light or a plurality of lights, the light(s) can, in some embodiments, be configured to illuminate a symbol indicative of the pump's orientation, such as a positive symbol (e.g., a plus sign, a smiling face, a check mark, etc.) indicative of the pump 20 being determined to be within the predefined range of predetermined acceptable orientations or a negative symbol (e.g., a minus sign, a frowning face, an “X,” etc.) indicative of the pump 20 being determined to not be within the predefined range of predetermined acceptable orientations. The pump 20 can include both the positive and negative symbols available for illumination or only one of the positive and negative symbols, which may help conserve power and/or help conserve space on the pump 20. In embodiments including a plurality of lights, there can be more than just the positive and negative symbols available for illumination. For example, a neutral face can be provided in addition to a smiling face and a frowning face, with illumination of the neutral face being indicative of the pump 20 being closer to an acceptable orientation than an unacceptable orientation. The user may therefore be informed that a direction in which the pump 20 is being moved is moving the pump 20 closer to the desired orientation by the frowning face ceasing to be illuminated and the neutral face becoming illuminated, or, conversely, that a direction in which the pump 20 is being moved is moving the pump 20 farther from the desired orientation by the neutral face ceasing to be illuminated and the frowning face becoming illuminated.

In some embodiments, whether a single light or a plurality of lights, the control circuitry 36 can be configured to turn off the light(s) in response to the start of drug delivery, e.g., in response to a “start” button being pushed on the pump 20, since the pump 20 is at the desired orientation. In other embodiments, whether a single light or a plurality of lights, the control circuitry 36 can be configured to turn off the light(s) in response to the end of drug delivery, which allows the user interface 40 to continue to indicate the pump's orientation during drug delivery so the user can use the orientation information provided via the user interface 40 to maintain the pump 20 at the desired orientation until drug delivery is complete. During the drug delivery, the control circuitry 36 can be configured to cause the light(s) to communicate information in addition to or instead of pump 20 orientation information, such as drug delivery progress information. For example, the control circuitry 36 can be configured to cause the light(s) to blink during drug delivery and to stop blinking and not be illuminated when drug delivery has completed. In other embodiments, whether a single light or a plurality of lights, the control circuitry 36 can be configured to turn off the light(s) in response to the end of the first pumping phase of the pumping assembly 26, which allows the user interface 40 to continue to indicate the pump's orientation during the first pumping phase so the user can use the orientation information provided via the user interface 40 to maintain the pump 20 at the desired orientation throughout the first pumping phase. As discussed above, the drug 22 moves from the reservoir 24 to the pump chamber 28 in the first pumping phase, while in the second pumping phase the drug 22 moves from the pump chamber 28 to the injector assembly 46 for delivery into the patient. In general, orientation of the pump 20 is more critical during the first pumping phase than during the second pumping phase since during the first pumping phase the conduit 38 needs to be in communication with the drug 22 in the reservoir 24 so the drug 22, but not air, enters the conduit 38 from the reservoir 24.

In another exemplary embodiment, the user interface 40 includes a vibrating mechanism configured to vibrate to indicate the pump's orientation. The vibration is configured to be felt by the patient wearing the pump 20. For example, the pump 20 can be configured to not provide any vibration signal (e.g., to not vibrate) until the pump 20 is determined to be within the predefined range of predetermined acceptable orientations, at which time the control circuitry 36 is configured to cause the vibrating mechanism to vibrate. For another example, the pump 20 can be configured to provide vibration via the vibrating mechanism until the pump 20 is determined to be within the predefined range of predetermined acceptable orientations, at which time the control circuitry 36 is configured to stop the vibration of the vibrating mechanism. For yet another example, the pump 20 can be configured to provide a first type of vibration (e.g., a slow vibration) until the pump 20 is determined to be within the predefined range of predetermined acceptable orientations, at which time the control circuitry 36 is configured to cause a second, different type of vibration (e.g., a faster vibration). For still another example, the vibrating mechanism can be configured as an electronic level with vibration of the vibrating mechanism being greater the farther the pump 20 is from the predefined range of predetermined acceptable orientations and the vibrating mechanism not vibrating when the pump 20 is determined to be within the predefined range of predetermined acceptable orientations. For another example, the pump 20 can be configured to not provide any vibration signal (e.g., to not vibrate) until the pump 20 is determined to be within a predetermined amount away from the predefined range of predetermined acceptable orientations, at which time the control circuitry 36 is configured to cause the vibrating mechanism to vibrate as a pre-notification of impending acceptable orientation.

In some embodiments in which the vibrating mechanism is configured to vibrate to indicate the pump 20 being determined to be within the predefined range of predetermined acceptable orientations, the control circuitry 36 can be configured to stop the vibration in response to the start of drug delivery, e.g., in response to a “start” button being pushed on the pump 20, since the pump 20 is at the desired orientation and so the vibration does not interfere with drug delivery. In other embodiments in which the vibrating mechanism is configured to vibrate to indicate the pump 20 being determined to be within the predefined range of predetermined acceptable orientations, the control circuitry 36 can be configured to turn off the vibrating mechanism in response to the end of drug delivery, which allows the user interface 40 to continue to indicate the pump's orientation during drug delivery so the user can use the orientation information provided via the user interface 40 to maintain the pump 20 at the desired orientation until drug delivery is complete. In other embodiments in which the vibrating mechanism is configured to vibrate to indicate the pump 20 being determined to be within the predefined range of predetermined acceptable orientations, the control circuitry 36 can be configured to turn off the vibrating mechanism in response to the end of the first pumping phase of the pumping assembly 26, which allows the user interface 40 to continue to indicate the pump's orientation during the first pumping phase so the user can use the orientation information provided via the user interface 40 to maintain the pump 20 at the desired orientation throughout the first pumping phase.

In another exemplary embodiment, the user interface 40 includes a speaker configured to provide an audio signal to provide an indication of the pump's orientation. The audio signal can be a single sound or a series of sounds. The sound(s) can be preprogrammed into the pump 20, e.g., into a memory of the control circuitry 36, and/or the sound(s) can be uploaded to the pump 20 by a user using a communication interface of the pump's control circuitry 36 that is configured to communicate (wired or wirelessly) with a device external to the pump 20. For example, the pump 20 can be configured to not provide any audio signal until the pump 20 is determined to be within the predefined range of predetermined acceptable orientations, at which time the control circuitry 36 is configured to cause an audio signal to be emitted from the speaker. For another example, the pump 20 can be configured to provide an audio signal until the pump 20 is determined to be within the predefined range of predetermined acceptable orientations, at which time the control circuitry 36 is configured to stop the sound (or series of sounds) from being emitted from the speaker. For yet another example, the pump 20 can be configured to provide a first type of audio signal (e.g., a sound or series of sounds having a first tone) until the pump 20 is determined to be within the predefined range of predetermined acceptable orientations, at which time the control circuitry 36 is configured to cause a second, different type of audio signal (e.g., a sound or series of sounds having a second tone) to be emitted from the speaker. For still another example, the audio signal can be configured as an electronic level with the audio signal being louder the farther the pump 20 is from the predefined range of predetermined acceptable orientations and no audio signal being provided when the pump 20 is determined to be within the predefined range of predetermined acceptable orientations.

In some embodiments in which an audio signal is provided to indicate that the pump 20 is determined to be within the predefined range of predetermined acceptable orientations, the control circuitry 36 can be configured to stop the audio signal in response to the start of drug delivery, e.g., in response to a “start” button being pushed on the pump 20, since the pump 20 is at the desired orientation. In other embodiments in which an audio signal is provided to indicate that the pump 20 is determined to be within the predefined range of predetermined acceptable orientations, the control circuitry 36 can be configured to turn off the audio signal in response to the end of drug delivery, which allows the user interface 40 to continue to indicate the pump's orientation during drug delivery so the user can use the orientation information provided via the user interface 40 to maintain the pump 20 at the desired orientation until drug delivery is complete. During the drug delivery, the control circuitry 36 can be configured to cause the speaker to communicate information in addition to or instead of pump 20 orientation information, such as drug delivery progress information. For example, the control circuitry 36 can be configured to cause the audio signal to be continuous to indicate that the pump 20 is determined to be within the predefined range of predetermined acceptable orientations and to be a sequential series of noises during drug delivery and to stop altogether when drug delivery has completed. In other embodiments in which an audio signal is provided to indicate that the pump 20 is determined to be within the predefined range of predetermined acceptable orientations, the control circuitry 36 can be configured to turn off the audio signal in response to the end of the first pumping phase of the pumping assembly 26, which allows the user interface 40 to continue to indicate the pump's orientation during the first pumping phase so the user can use the orientation information provided via the user interface 40 to maintain the pump 20 at the desired orientation throughout the first pumping phase.

In another exemplary embodiment, the user interface 40 includes a display configured to show thereon an indication of the pump's orientation. The display can include a display screen having any of a variety of configurations, such as a cathode ray tube (CRT), a liquid crystal display (LCD), a touchscreen, etc. For example, text shown on the display can indicate whether or not the pump 20 is determined to be within the predefined range of predetermined acceptable orientations. For another example, similar to the light(s) discussed above, colors and/or symbols shown on the display can indicate whether or not the pump 20 is determined to be within the predefined range of predetermined acceptable orientations. For yet another example, a digital level shown on the display, similar to the electronic level of lights discussed above, can indicate whether or not the pump 20 is determined to be within the predefined range of predetermined acceptable orientations.

The display can be located on a same side of the pump 20 as a “start” button of the pump 20 that is configured to be depressed by a user to begin drug delivery from the pump 20. The user may therefore be less likely to not see information shown on the display before beginning drug delivery since the display is located on a side of the pump 20 that the user will tend to look at when pushing the “start” button.

In some embodiments in which a display is provided to indicate that the pump 20 is determined to be within the predefined range of predetermined acceptable orientations, the control circuitry 36 can be configured to stop providing pump 20 orientation information via the display in response to the start of drug delivery, e.g., in response to a “start” button being pushed on the pump 20, since the pump 20 is at the desired orientation. In other embodiments in which a display is provided to indicate that the pump 20 is determined to be within the predefined range of predetermined acceptable orientations, the control circuitry 36 can be configured to stop providing pump 20 orientation information via the display in response to the end of drug delivery, which allows the user interface 40 to continue to indicate the pump's orientation during drug delivery so the user can use the orientation information provided via the user interface 40 to maintain the pump 20 at the desired orientation until drug delivery is complete. During the drug delivery, the control circuitry 36 can be configured to cause the display to communicate information in addition to or instead of pump 20 orientation information, such as drug delivery progress information. For example, the control circuitry 36 can be configured to cause the display to show information indicating that drug delivery is occurring and/or to show a progress bar indicating progress of the drug delivery. In other embodiments in which a display is provided to indicate that the pump 20 is determined to be within the predefined range of predetermined acceptable orientations, the control circuitry 36 can be configured to stop providing pump 20 orientation information via the display in response to the end of the first pumping phase of the pumping assembly 26, which allows the user interface 40 to continue to indicate the pump's orientation during the first pumping phase so the user can use the orientation information provided via the user interface 40 to maintain the pump 20 at the desired orientation throughout the first pumping phase.

In another exemplary embodiment, the user interface 40 includes a mechanical level configured to indicate the pump's orientation. The mechanical level does not require power to operate and may therefore always be functional and may allow a smaller and/or less powerful power supply to be used with the pump 20. The mechanical level is configured and used similar to the electronic level discussed above but is implemented mechanically, e.g., using one bubble in a container filled with a liquid. The bubble is visible through a transparent or semi-transparent wall of the container. The pump 20 being at the desired orientation for drug delivery corresponds to the bubble being aligned with a marker on the container. Mechanical levels are familiar to many people, so the mechanical level may thus be easy for the user to understand. In an exemplary embodiment, the mechanical level is a single mechanical level, which may help reduce cost of the pump 20.

The pump 20 can include a single type of user interface 40, such as only including a light as the user interface 40, only including a vibrating mechanism as the user interface 40, only including a speaker as the user interface 40, only including a display as the user interface 40, or only including a mechanical level as the user interface 40. Alternatively, the pump 20 can include more than one type of user interface 40, e.g., including a light and a speaker, including a speaker and a vibrating mechanism, including a display and a speaker, including a display and a vibrating mechanism, including a light, a vibrating mechanism, and a speaker, including a mechanical level and a speaker, etc. Providing more than one type of user interface 40 may increase an initial cost of the pump 20 but may provide redundancy and thereby help save costs in the long run and/or increase the likelihood that the pump 20 is in an acceptable orientation for drug delivery when drug delivery begins because the user has the opportunity to observe more than one indicator of pump orientation. Some users may have difficulty observing a certain type of indicator, such as by a user being hard of hearing and thus having difficulty detecting an audio signal, a user having impaired eyesight and thus having difficulty seeing a display, or a user being colorblind and having difficulty discerning different colored lights or different colors on a display, so providing multiple types of user interfaces 40 may be particularly helpful for those users.

In embodiments in which the user interface 40 includes multiple types of user interfaces 40, the pump 20 can be configured to allow one or more of the user interfaces 40 to be disabled as long as at least one other of the user interfaces 40 remains enabled. In this way, a user can disable user interface(s) 40 that would not be useful, such if a user is hard of hearing and thus would not be helped by an audio signal, a user having impaired eyesight and thus not likely or able to read a display, a user being in a dark location such as a movie theater and temporarily not wanting light(s) to illuminate, a user being in a location where sound would be distracting to others such as in a movie theater, business meeting, or school lecture and thus temporarily not wanting audio to sound, etc. User interfaces can be configured to be disabled in any of a variety of ways. For example, when at least one of the user interfaces 40 includes a display as a touchscreen, input to the touchscreen can select which one or more user interfaces 40 to disable, with the touchscreen not being an option for disabling to provide continual ability to provide touch inputs to the pump 20. For another example, when at least one of the user interfaces 40 includes a light, the pump 20 can include a manually operable switch configured to allow the light to be selectively enabled for illumination and disabled for illumination. For another example, when at least one of the user interfaces 40 includes a speaker, the pump 20 can include a manually operable switch configured to allow the speaker to be selectively enabled to provide audio (unmuted) and disabled from providing audio (muted).

Regardless of the type of the user interface 40 and whether or not the user interface 40 includes one type or multiple types of user interfaces 40, the user interface 40 can be configured to provide one or more types of information in addition to pump 20 orientation information. For example, the user interface 40 can be configured to indicate an error state of the pump 20, such as low power supply, improper needle advancement into the patient, incompatible type of reservoir 24 loaded into the pump housing, etc. The error state can be communicated in a variety of ways depending on the type of the user interface 40. For example, when the user interface 40 includes light(s), a particular light color and/or light pattern can be a predetermined indicator of an error state. Different light colors and/or light patterns can be indicative of different error states. For another example, when the user interface 40 includes a vibration mechanism, a particular vibration pattern can be a predetermined indicator of an error state. Different vibration patterns can be indicative of different error states. For yet another example, when the user interface 40 includes a speaker, a particular sound or series of sounds can be a predetermined indicator of an error state. Different sounds or series of sounds can be indicative of different error states. For another example, when the user interface 40 includes a display, text can be provided to identify an error state. Different text can be indicative of different error states. For still another example, when the user interface 40 includes a display, a particular symbol can be provided to identify an error state. Different symbols can be indicative of different error states. For another example, when the user interface 40 includes at least two types of user interfaces, different combinations of information provided by the multiple user interfaces can indicate different pump 20 states, such as a particular combination of light color and sound indicating a state, a particular combination of light color, vibration, and sound indicating a state, a particular combination of vibration and sound indicating a state, etc.

The various possible pump states that can be indicated by the user interface 40 can be provided for user reference in the pump's written instructions (in paper and/or online) and/or printed on the pump 20. Examples of pump states include the pump 20 being ready to begin drug delivery, the pump 20 having completed drug delivery, the pump 20 needing its power supply to be replaced, all drug doses having been delivered from the reservoir 24 such that a new reservoir 24 and/or new pump 20 is needed for continued administration of the drug to the patient, the pump 20 being initially powered on, etc.

Regardless of the type of the user interface 40 and whether or not the user interface 40 includes one type or multiple types of user interfaces 40, the user interface 40 providing an indication as to whether or not the pump 20 is in an acceptable orientation for drug delivery to begin allows the patient to be reoriented until the pump 20 is in the desired orientation for drug delivery. The patient can be reoriented by changing their position, e.g., standing, sitting, lying down on their left side, lying down on their right side, etc., with or without assistance from another person. In other words, the pump 20 is configured to be reoriented via patient movement, e.g., movement of the patient causes movement of the pump 20. In some embodiments, the pump 20 can also be configured to be reoriented via movement of the pump 20 relative to the patient. In such embodiments, the user interface 40 providing an indication as to whether or not the pump 20 is in an acceptable orientation for drug delivery to begin allows the pump 20 to be reoriented relative to the patient until the pump 20 is in the desired orientation for drug delivery. The pump 20 can be configured to be reoriented via movement of the pump 20 relative to the patient by being releasably attachable to the patient such that the pump 20 can be attached to the patient, removed therefrom, and re-attached thereto any number of times until the pump 20 is desirably oriented.

The pump 20 can be configured to prevent drug delivery until the pump 20 is in an acceptable orientation for drug delivery to begin, which may help ensure that the drug is successfully delivered to the patient. The control circuitry 36 can be configured to prevent drug delivery until the control circuitry 36 determines that the pump 20 is in an acceptable orientation for drug delivery to begin. For example, the control circuitry 36 can be configured to not activate the pumping assembly 26 such as by the control circuitry 36 not responding to depression of a start button on the pump 20, until determining that the pump 20 is in an acceptable orientation for drug delivery to begin. For another example, the pump 20 can include a switch movable between a first position, in which the pumping assembly 26 cannot be activated to begin drug delivery, and a second position, in which the pumping assembly 26 can be activated to begin drug delivery. The switch can be in the first position until the control circuitry 36 determines that the pump 20 is in an acceptable orientation for drug delivery to begin, at which time the control circuitry 26 can cause the switch to move from the first position to the second position.

FIG. 4 illustrates an embodiment of the pump 20 that includes a housing 21 and a backing or label 23 configured to be removed from the housing 21 to expose adhesive attachable to the patient. With the pump 20 adhered to the patient via the adhesive, the pump 20 is configured to be reoriented via patient movement in response to information provided via the user interface 40.

FIGS. 5-7 illustrate an embodiment of the pump 20 configured to be reoriented via movement of the pump 20 relative to a patient wearing the pump 20. The pump 20 includes a housing 25, a housing mount 27 releasably attached to the housing 25, and a backing or label 29 configured to be removed from the housing mount 27 to expose adhesive attachable to the patient. With the pump 20 adhered to the patient via the adhesive, the housing 25 is configured to be removed from the housing mount 27 adhered to the patient and reattached to the housing mount 27 adhered to the patient. In this way, the pump 20 can be reoriented relative to the patient in response to information provided via the user interface 40. FIG. 5 illustrates the pump 20 prior to attachment of the pump 20 to the patient. FIG. 6 shows the housing mount 27 of the pump 20 adhered to a skin surface 31 of a patient and the housing 25 removed from the housing mount 27. FIG. 7 shows the housing 25 reattached to the housing mount 27 at a different orientation relative to the housing mount 27 than in the pump's initial configuration (shown in FIG. 5).

The housing mount 27 includes a Velcro surface 27s configured to releasably engage a corresponding Velcro surface (obscured in FIGS. 5-7) of the housing 27. The Velcro surfaces allow the housing 25 to be reoriented relative to the housing mount 27, and hence to the patient to which the housing mount 27 is adhered, at any selected orientation. Although Velcro is used to releasably attach the housing 25 and housing mount 27 in this illustrated embodiment, other releasable attachment mechanisms can be used, such as a magnet on a surface of one of the housing 25 and housing mount 27 and a magnetic surface on the other of the housing 25 and housing mount 27, or a ball joint mechanism in which one of the housing 25 and housing mount 27 includes a ball and the other of the housing 25 and housing mount 27 includes a socket configured to releasably seat the ball therein.

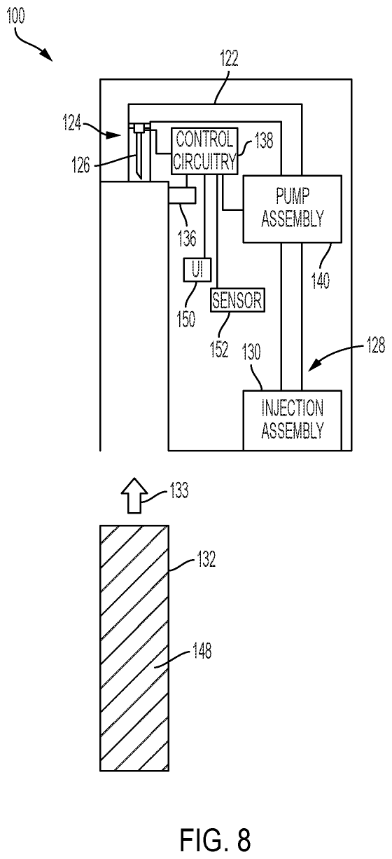

FIGS. 8-10 illustrate another embodiment of a pump 100 configured to be worn by a patient and to deliver a drug 148 to the patient. The pump 100 of FIGS. 8-10 is generally configured and used similar to the pump 20 of FIG. 1. The pump 100 is configured to engage with a prefilled therapeutic substance reservoir 132. Within the pump 100 is a sterile fluid path 122 for delivering a drug 148 from the reservoir 132 to a patient wearing the pump 100. The sterile fluid path 122 has a conduit 126 at an upstream end 124 of the sterile fluid path 122 and has an injection assembly (also referred to herein as an “injector assembly”) 130 at a downstream end 128 of the sterile fluid path 122. The pump 100 also includes a user interface (UI) 150 configured to indicate an orientation of the pump 100 to a user of the pump, and a sensor 152 configured to monitor an orientation of the pump 100 relative to gravity, e.g., the ground.

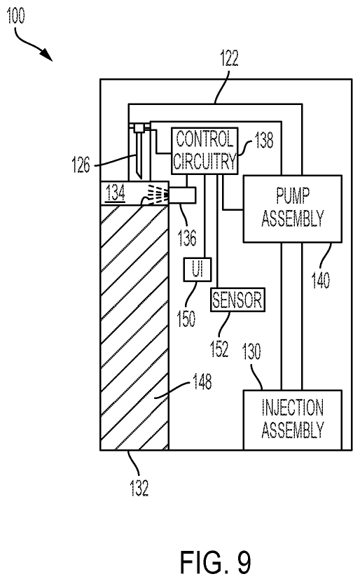

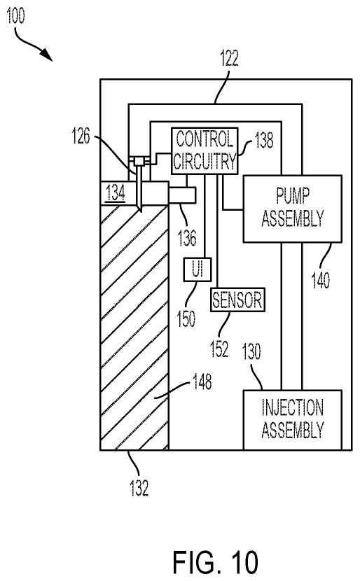

The pump 100 and the prefilled therapeutic substance reservoir 132 are configured to engage with one another, such as shown by arrow 133 in FIG. 8, e.g., the reservoir 132 is configured to be inserted into the pump 100. When the pump 100 and the reservoir 132 are engaged with one another, such as is shown in FIG. 9, a sealed disinfection chamber 134 is defined between the sterile fluid path 122 and the reservoir 132. While the pump 100 and the reservoir 132 are typically sterile, the disinfection chamber 134 is (a) initially non-sterile, and (b) typically sealed from further bacteria or virus penetration. The conduit 126 is configured to be driven to penetrate the disinfection chamber 134 and subsequently the reservoir 132 when the pump 100 and the reservoir 132 are engaged with one another, such that fluid communication is established between the reservoir 132 and the sterile fluid path 122, such as is shown in FIG. 10.

The pump 100 includes a disinfection assembly 136 configured to disinfect the disinfection chamber 134 prior to the conduit 126 penetrating the disinfection chamber 134 and thus before the conduit 126 enters the reservoir 132. The pump 100 includes control circuitry 138 configured to activate the disinfection assembly 136, to subsequently terminate the activation of the disinfection assembly 136, and to then drive the conduit 126 to penetrate the disinfection chamber 134 and subsequently the reservoir 132.

Once fluid communication is established between the reservoir 132 and the sterile fluid path 122, the control circuitry 138 is configured to drives a pump assembly 140 to draw the drug 148 from the reservoir 132 and deliver it to the patient via injection assembly 130 similar to that discussed above regarding the control circuitry 36 and the injector assembly 46 of FIG. 1.

The user interface 150 can have any of a variety of configurations, such as any one or more of the user interfaces discussed above with respect to the user interface 40 of FIG. 1.

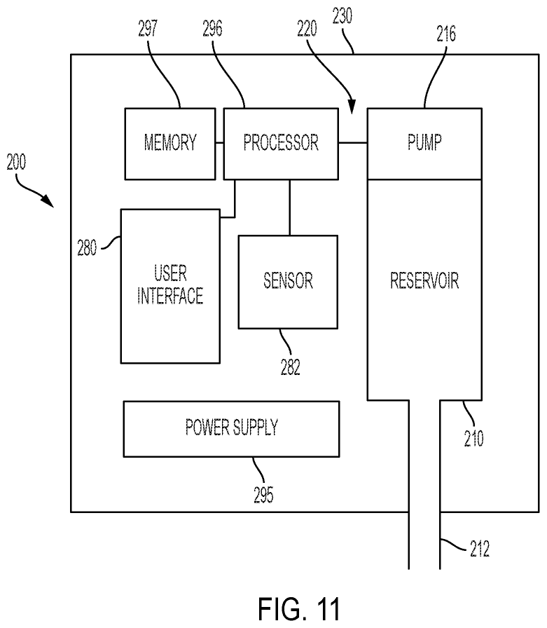

FIG. 11 illustrates another embodiment of a pump 200 configured to be worn by a patient and to deliver a drug to the patient. The pump 200 of FIG. 11 is generally configured and used similar to the pump 20 of FIG. 1. The pump 200 includes a reservoir 210 configured to contain a liquid drug therein to be delivered from the pump 200. The pump 200 also includes a pumping assembly 216 configured to cause dispensing of the drug contained in the reservoir 210 so that the drug can be delivered to the patient. The pump 200 also includes an injector assembly that includes an infusion line 212, e.g., a needle or a cannula. The drug is delivered from the reservoir 210 upon actuation of the pumping assembly 216 via the infusion line 212.

The pump 200 also includes a user interface 280 configured to indicate an orientation of the pump 200 to a user of the pump, and a sensor 282 configured to monitor an orientation of the pump 200 relative to gravity, e.g., the ground. The user interface 280 can have a variety of configurations, such as any one or more of the user interfaces discussed above with respect to the user interface 40 of FIG. 1.

The pump 200 also includes control circuitry that includes a processor 296 and a memory 297 in operative communication with the processor 296. Actuation of the pumping assembly 216 is controlled by the processor 296, which is in operative communication with the pumping assembly 216 for controlling the pump's operation.

In at least some embodiments, the processor 296 is configured to be programmed by a user, e.g., the patient, a healthcare professional, etc., via the user interface 280. The processor 296 being user-programmable enables the pump 200 to deliver the drug to the patient in a controlled manner specific to the patient. The user can enter parameters, such as infusion duration and delivery rate, via the user interface 280, such as by the user interface 280 including a touchscreen configured to receive touch input thereto, the user interface 280 including selector button(s), and/or the user interface 280 including a keypad. The delivery rate can be set by the user to a constant infusion rate or as set intervals for periodic delivery, typically within pre-programmed limits. The programmed parameters for controlling the pumping assembly 216 are stored in and retrieved by the processor 296 from the memory 297.

The pump 200 also includes a power supply 295 configured to provide power to any components of the pump 200 that require power for operation, such as the pumping assembly 216, the processor 296, the user interface 280, and the sensor 282.

The reservoir 210, the pumping assembly 216, the user interface 280, the power supply 295, the processor 296, and the memory 297 are located within a housing (also referred to herein as a “body” of a pump) 230 of the pump 200. The infusion line 212 is partially located within the housing 230 and extends from the housing 230 for penetration into the patient. The infusion line 212 can be fixedly positioned partially within the housing 230 and partially outside the housing 230, as shown in FIG. 11, or the infusion line 212 can be movable, e.g., under control of the circuitry, from an initial position entirely within the housing 230 to a delivery position partially within the housing 230 and partially outside the housing 230.

In embodiments in which the user interface 280 is visual, e.g., by including one or more lights and/or including a display, the user interface 280 can be located on a portion of the housing 230 that is angled to facilitate the patient's visualization of the user interface 280 while the pump 200 is attached to the patient in accordance with instructions indicating how the pump 200 should be attached to the patient. The angled portion of the housing 230 that includes the user interface 280 thereon may also help ensure that the pump 200 is attached to the patient in accordance with instructions indicating how the pump 200 should be attached to the patient because the angled portion 230 being angled in a way so as to obscure some or all of the user interface 280 from the patient's view can serve as a clue that the pump 200 is not yet in its recommended attachment orientation.

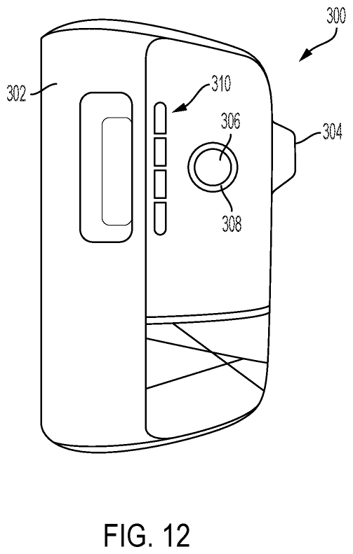

FIG. 12 illustrates another embodiment of a pump 300 configured to be worn by a patient and to deliver a drug to the patient. The pump 300 of FIG. 12 is generally configured and used similar to the pump 20 of FIG. 1, e.g., includes a body 302, a removable backing or label 304, a depressible button 306, a user interface, a reservoir configured to contain a liquid drug therein to be delivered from the pump 300, a pumping assembly configured to cause dispensing of the drug contained in the reservoir, an injector assembly configured to deliver the drug into the patient, a sensor configured to monitor an orientation of the pump 300 relative to gravity, a plunger configured to slide within the pump chamber, and control circuitry operatively connected to the sensor, the user interface, and the pumping assembly. As discussed above, the reservoir can be prefilled by a medical vendor or can be otherwise filled, and the reservoir can be preloaded into the pump 300 or can be loaded therein by a user. The reservoir, pumping assembly, injector assembly, sensor, plunger, and control circuitry are obscured by the body 302 in FIG. 12.

The user interface in this illustrated embodiment includes a plurality of lights including a first light 308 and a second light 310. The first light 308 is a single light extending circumferentially around an entire perimeter of the button 306. The second light 310 is a series of lights arranged in a line. The second light 310 includes four lights in this illustrated embodiment but can include another plural number of lights. The first and second lights 308, 310 are off (unilluminated) in FIG. 12.

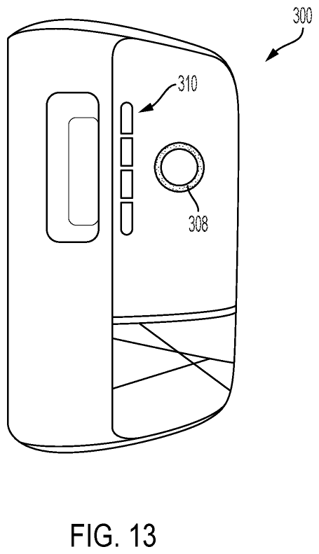

FIG. 13 shows the first light 308 on (illuminated) in a first color (e.g., green or other color) and the second light 310 off. The control circuitry can be configured to cause the first light 308 to be illuminated as in FIG. 13 in response to the manual removal of the backing 304 or after the backing 304 has been removed and the pump 300 adhered to a patient's skin. The pump 300 can include a sensor configured to monitor whether the backing 304 is on the pump 300 or has been removed therefrom, such as a pressure sensor configured to monitor pressure applied thereto by the backing 304 or a light sensor configured to monitor light received thereby with the backing 304 preventing the light sensor from receiving light until removal of the backing 304 from the housing 302. The pump 300 can include a sensor configured to monitor whether the pump 300 has been adhered to the patient's skin, such as a pressure sensor configured to have pressure applied thereto by a lever or switch pushed thereagainst in response to the pump 300 being adhered to the patient's skin. In some embodiments, the control circuitry can be configured to cause the first light 308 to blink in the first color in response to the manual removal of the backing 304 and then be unilluminated after the pump 300 has been adhered to a patient's skin. Alternatively, the control circuitry can be configured to cause the first light 308 to be continuously illuminated in the first color in response to the manual removal of the backing 304 and then blink in the first color after the pump 300 has been adhered to a patient's skin.

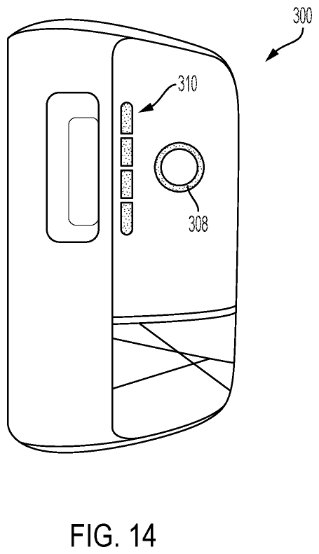

FIG. 14 shows the first light 308 on in the first color and the second light 310 on in the first color. The control circuitry can be configured to cause the second light 310 to be illuminated (either continuously or blinking) during priming. The first light 308 is on at this time (either continuously or blinking, as discussed above). The pump 300 can include a speaker (obscured in the figures) configured to provide an audio signal, e.g., one beep, a series of two beeps, a series of three beeps, a musical sequence, etc., as controlled by the control circuitry, when priming has been completed and prior to needle or cannula insertion into the patient. The audio signal can thereby indicate to the user that needle or cannula insertion is imminent.

Depression of the button 306 is configured to start drug delivery. In an exemplary embodiment, the first and second lights 308, 310 are configured to indicate to the user that pump 300 is ready to begin drug delivery by both of the first and second lights 308, 310 being illuminated in the first color in a continuous manner. The first light 308 is configured to indicate an orientation of the pump 300 to the user as discussed above, e.g., by being illuminated when the pump 300 is determined by the control circuitry to be is in an acceptable orientation for drug delivery based on data from the sensor monitoring pump 300 orientation. The second light 310 is configured to indicate drug delivery progress. FIG. 14 illustrates the pump 300 in a configuration in which the first and second lights 308, 310 are on to indicate that the pump 300 is ready to begin drug delivery.

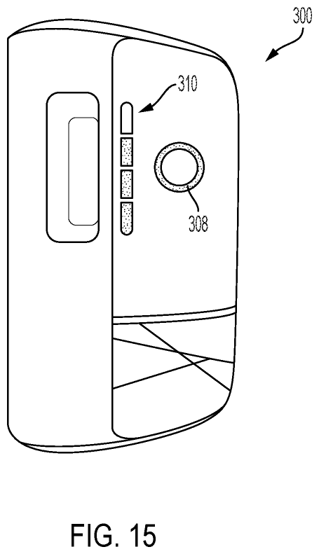

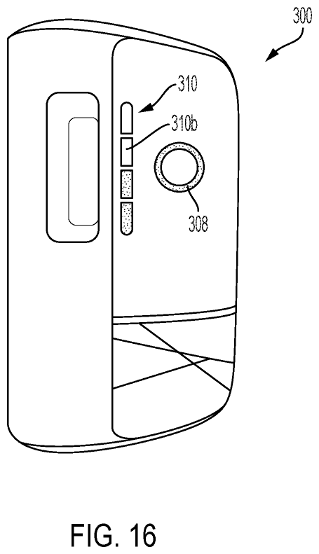

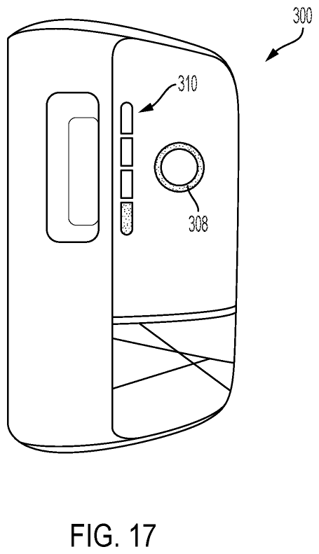

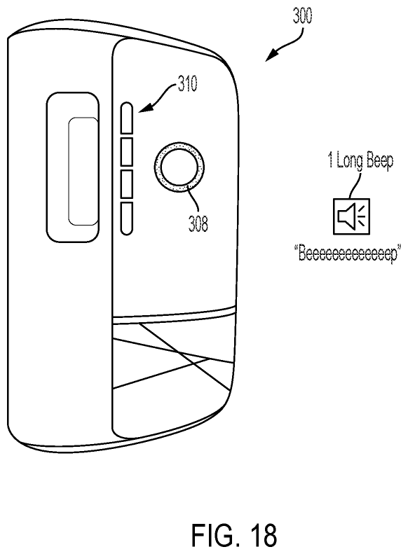

FIGS. 15-18 indicate a progress of drug delivery as indicated by the second light 310. The first light 308 remains in the illuminated state of FIG. 14 in FIGS. 15-18. The second light 310 acts as a countdown mechanism with lights in the series of lights being sequentially turned off as drug delivery occurs to indicate the drug delivery's progress. When drug delivery begins, e.g., when the button 306 is pressed, all four of the lights of the second light 310 are on. When drug delivery is about 25% complete, as determined by the control circuitry, an uppermost one of the light series of the second light 310 is turned off by the control circuitry such that three quarters of the light series is on and one quarter of the light series is off, as shown in FIG. 15. When drug delivery is about 50% complete, as determined by the control circuitry, a second uppermost one 310b of the light series of the second light 310 is turned off by the control circuitry such that half of the light series is on and half the light series is off, as shown in FIG. 16. When drug delivery is about 75% complete, as determined by the control circuitry, a third uppermost one of the light series of the second light 310 is turned off by the control circuitry such that one quarter of the light series is on and three quarters of the light series is off, as shown in FIG. 17. When drug delivery is complete, as determined by the control circuitry, the fourth one of the light series of the second light 310 is turned off by the control circuitry such that all of the light series is off, as shown in FIG. 18. FIG. 18 also illustrates an audio signal being provided to also indicate that drug delivery is complete. The audio signal is one long beep in this illustrated embodiment, but another audio signal can be used to indicate end of drug delivery. In other embodiments with the second light 310 including a plural number of lights other than four, the lights will be sequentially turned off at different percentages of drug delivery completion.

In the event that an error occurs during drug delivery, one or both of the first and second lights 308, 310 can be configured to indicate occurrence of the error as determined by the control circuitry according to pre-programmed instructions. Examples of errors include the orientation of the pump 300 changing from the acceptable orientation for drug delivery to an unacceptable orientation for drug delivery, occlusion in the drug fluid path in the pump 300, power loss, etc.

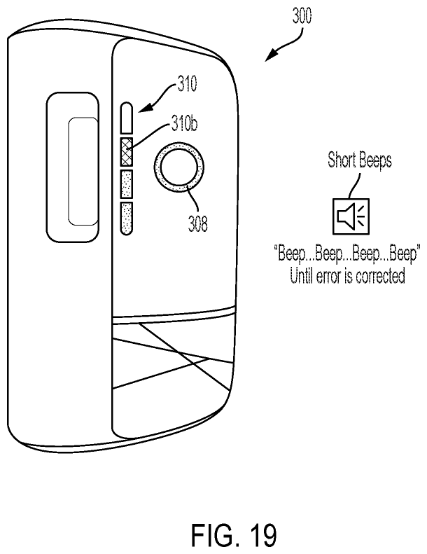

In an exemplary embodiment, the second light 310 is configured to indicate occurrence of an error during drug delivery by changing from the first color to a second color (e.g., red or other color). Which one of the series of lights in the second light 310 changes from the first color to the second color indicates a general timing of when the error occurred during drug delivery. For example, if an error occurs after about 75% of drug delivery is complete but before about 50% of drug delivery has been completed, e.g., when the second light 310 is illuminated as shown in FIG. 15, the second uppermost one 310b of the light series, which is the uppermost illuminated one of the series of lights, is changed from the first color to the second color by the control circuitry, as shown in FIG. 19. FIG. 19 also illustrates an audio signal being provided to also indicate that an error occurred. In this illustrated embodiment, the audio signal is a series of short beeps that continue until the error is corrected, but another audio signal can be used to indicate error occurrence. An example of a correctable error is the pump 300 being in an unacceptable orientation for drug delivery.

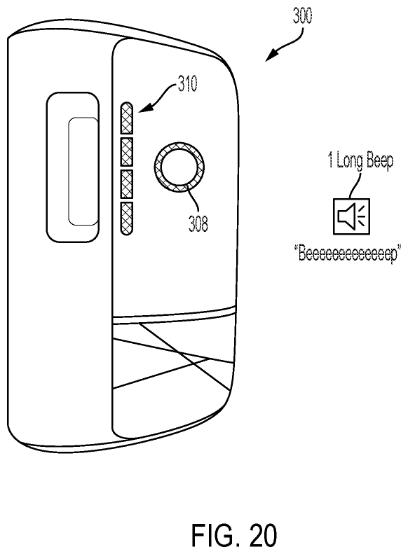

If an error is determined to have occurred that cannot be corrected, a so-called “fatal error,” each of the first and second lights 308, 310 can be configured to indicate occurrence of the fatal error. Examples of fatal errors include occlusion in the drug fluid path in the pump 300, no drug remaining in the reservoir and the reservoir not being configured for refill or replacement, and the pump's power supply being depleted of power. For example, as shown in FIG. 20, the control circuitry can be configured to cause each of the first and second lights 308, 310 to be in the second color in response to occurrence of a fatal error. FIG. 20 also illustrates an audio signal being provided to also indicate that a fatal error occurred. In this illustrated embodiment, the audio signal is continuous long beep, but another audio signal can be used to indicate fatal error occurrence.

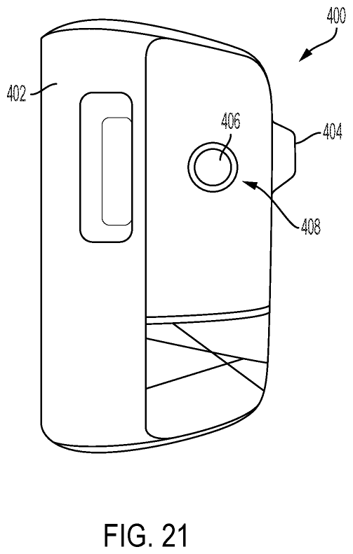

FIG. 21 illustrates another embodiment of a pump 400 configured to be worn by a patient and to deliver a drug to the patient. The pump 400 of FIG. 21 is generally configured and used similar to the pump 20 of FIG. 1, e.g., includes a body 402, a removable backing or label 404, a depressible button 406, a user interface, a reservoir configured to contain a liquid drug therein to be delivered from the pump 400, a pumping assembly configured to cause dispensing of the drug contained in the reservoir, an injector assembly configured to deliver the drug into the patient, a sensor configured to monitor an orientation of the pump 400 relative to gravity, a plunger configured to slide within the pump chamber, and control circuitry operatively connected to the sensor, the user interface, and the pumping assembly. As discussed above, the reservoir can be prefilled by a medical vendor or can be otherwise filled, and the reservoir can be preloaded into the pump 400 or can be loaded therein by a user. The reservoir, pumping assembly, injector assembly, sensor, plunger, and control circuitry are obscured by the body 402 in FIG. 21.

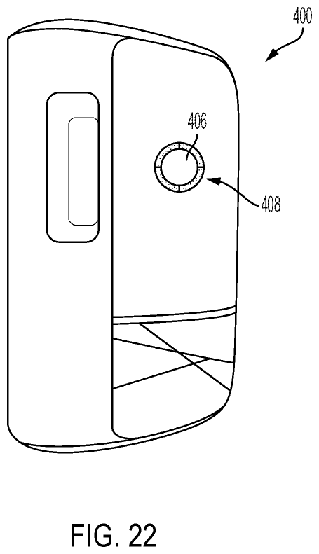

The user interface in this illustrated embodiment includes a plurality of lights, namely a light 408 that includes a series of lights arranged circumferentially around an entire perimeter of the button 406. The light 408 includes four lights in this illustrated embodiment but can include another plural number of lights. FIG. 21 shows the light 408 off. FIG. 22 shows all four lights of the light 408 illuminated in a first color (e.g., green or other color), having been turned on by the control circuitry in response to removal of the backing 404 or adherence of the pump 400 to the patient's skin similar to that discussed above regarding the pump 300.

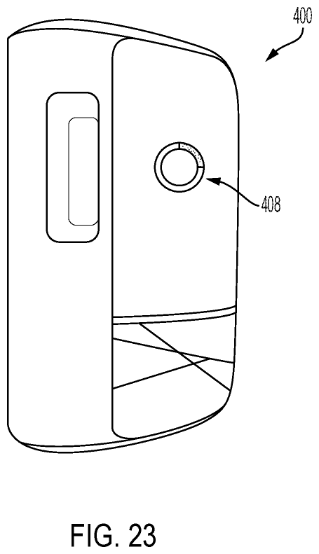

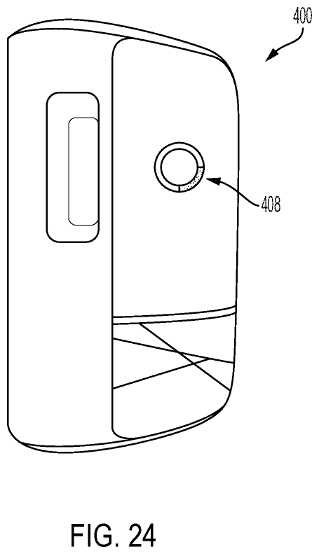

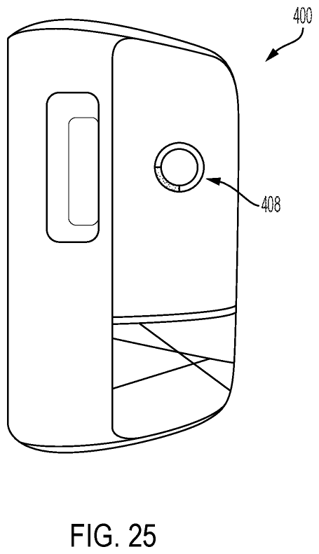

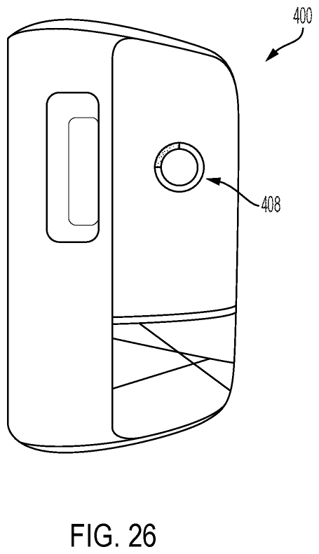

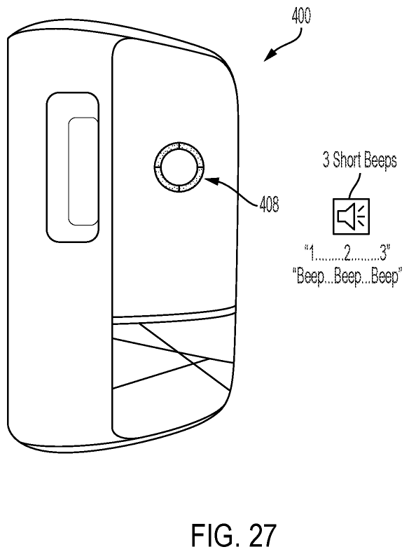

The control circuitry can be configured to cause the light 408 to be illuminated during priming. In an exemplary embodiment, each of the lights in the series of lights is illuminated sequentially around the button's perimeter during priming to indicate that priming is occurring, as shown in FIGS. 23-26. As shown in FIG. 27, the light 408 can be fully illuminated (all of the four lights on, either blinking or on continuously) to indicate completion of priming. The pump 400 can include a speaker (obscured in the figures) configured to provide an audio signal, e.g., one beep, a series of two beeps, a series of three beeps, a musical sequence, etc., as controlled by the control circuitry, when priming has been completed and prior to needle or cannula insertion into the patient. The audio signal can thereby indicate to the user that needle or cannula insertion is imminent. FIG. 27 illustrates the audio signal as a series of three short beeps.

Depression of the button 406 is configured to start drug delivery. The light 408 is configured to indicate an orientation of the pump 400 to the user as discussed above, e.g., by being illuminated (all lights in the series) when the pump 400 is determined by the control circuitry to be in an acceptable orientation for drug delivery based on data from the sensor monitoring pump 400 orientation. FIG. 27 illustrates the pump 400 in a configuration in which the light 408 indicates that the pump 400 is ready to begin drug delivery.

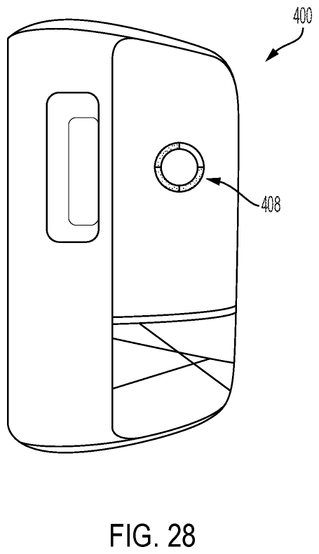

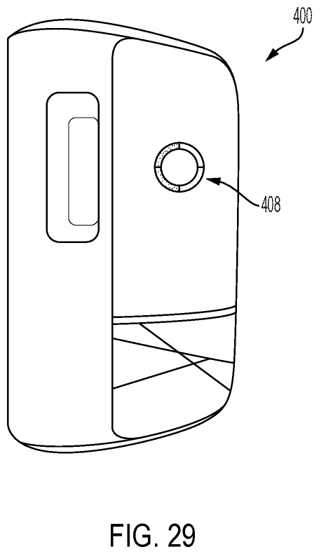

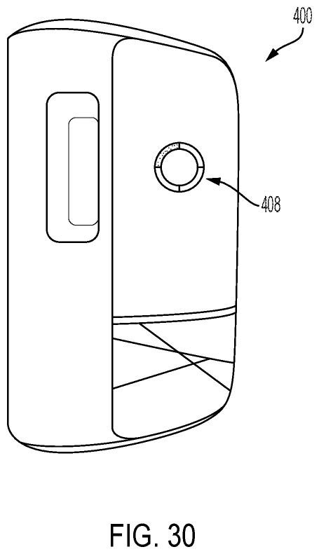

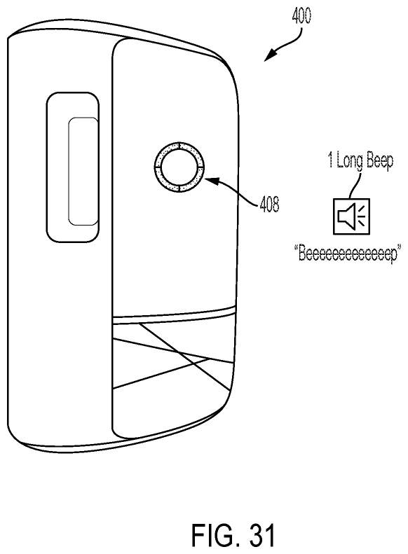

FIGS. 28-31 indicate a progress of drug delivery as indicated by the light 408. The drug delivery progress indicated by the light 408 is similar to that indicated by the second light 310 of the pump 300 by acting as a countdown mechanism with the series of lights being sequentially turned off as drug delivery occurs to indicate the drug delivery's progress. When drug delivery begins, e.g., when the button 406 is pressed, all four of the lights of the light 408 are on, as shown in FIG. 27. When drug delivery is about 25% complete, as determined by the control circuitry, one of the lights is turned off by the control circuitry such that three quarters of the light series is on and one quarter of the light series is off, as shown in FIG. 28. A top upper right one of the lights is off in this illustrated embodiment, but another one of the lights can be the first one turned off during drug delivery. When drug delivery is about 50% complete, as determined by the control circuitry, a second one of the light series is turned off by the control circuitry such that half of the light series is on and half the light series is off, as shown in FIG. 29. The lights are turned off in a clockwise manner in this illustrated embodiment, but the lights can instead be turned off in a counterclockwise manner. When drug delivery is about 75% complete, as determined by the control circuitry, a third one of the light series is turned off by the control circuitry such that one quarter of the light series is on and three quarters of the light series is off, as shown in FIG. 30. When drug delivery is complete, as determined by the control circuitry, the last one of the light series is turned off by the control circuitry such that all of the light series is off, as shown in FIG. 31. FIG. 31 also illustrates an audio signal being provided, e.g., via a speaker of the pump 400 as controlled by the control circuitry, to also indicate that drug delivery is complete. The audio signal is one long beep in this illustrated embodiment, but another audio signal can be used to indicate end of drug delivery. In other embodiments with the light 408 including a plural number of lights other than four, the lights will be sequentially turned off at different percentages of drug delivery completion.

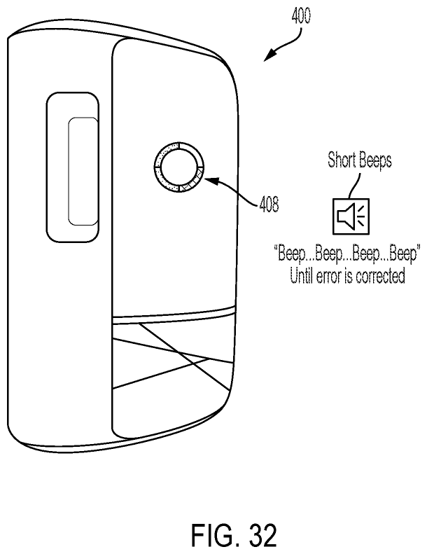

In the event that an error occurs during drug delivery, the light 408 can be configured to indicate occurrence of the error. In an exemplary embodiment, the light 408 is configured to indicate occurrence of an error during drug delivery by at least one of the series of lights changing from the first color to a second color (e.g., red or other color). Which one of the series of lights changes from the first color to the second color indicates a general timing of when the error occurred during drug delivery. For example, if an error occurs after about 75% of drug delivery is complete but before about 50% of drug delivery has been completed, e.g., when the light 408 is illuminated as shown in FIG. 28, the second one of the light series that would be turned off is changed from the first color to the second color by the control circuitry, as shown in FIG. 32. FIG. 32 also illustrates an audio signal being provided to also indicate that an error occurred. In this illustrated embodiment, the audio signal is series of short beeps that continue until the error is corrected, but another audio signal can be used to indicate error occurrence.

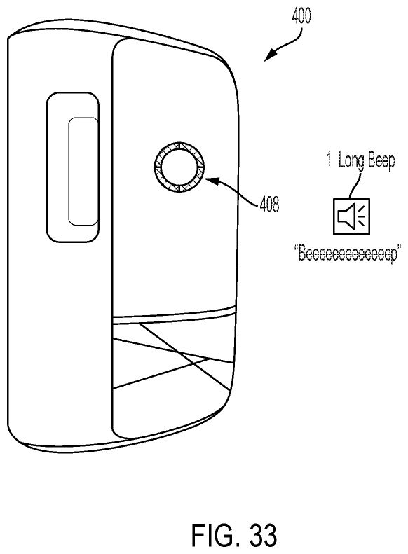

If a fatal error is determined to have occurred, e.g., as determined by the control circuitry, the light 408 can be configured to indicate occurrence of the fatal error. For example, as shown in FIG. 33, the control circuitry can be configured to cause the light 408 to be in the second color in response to occurrence of a fatal error. FIG. 33 also illustrates an audio signal being provided to also indicate that a fatal error occurred. In this illustrated embodiment, the audio signal is continuous long beep, but another audio signal can be used to indicate fatal error occurrence.

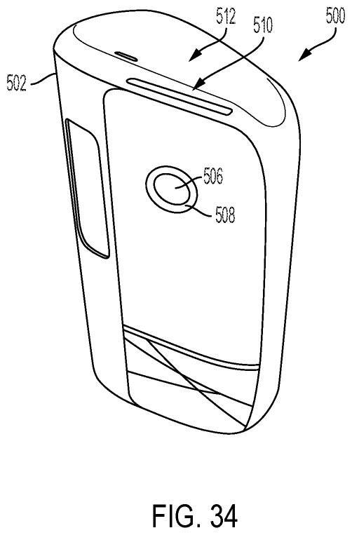

FIG. 34 illustrates another embodiment of a pump 500 configured to be worn by a patient and to deliver a drug to the patient. The pump 500 of FIG. 34 is generally configured and used similar to the pump 20 of FIG. 1, e.g., includes a body 502, a removable backing or label (obscured in FIG. 34), a depressible button 506, a user interface, a reservoir configured to contain a liquid drug therein to be delivered from the pump 500, a pumping assembly configured to cause dispensing of the drug contained in the reservoir, an injector assembly configured to deliver the drug into the patient, a sensor configured to monitor an orientation of the pump 500 relative to gravity, a plunger configured to slide within the pump chamber, and control circuitry operatively connected to the sensor, the user interface, and the pumping assembly. As discussed above, the reservoir can be prefilled by a medical vendor or can be otherwise filled, and the reservoir can be preloaded into the pump 500 or can be loaded therein by a user. The reservoir, pumping assembly, injector assembly, sensor, plunger, and control circuitry are obscured by the body 502 in FIG. 34. FIG. 34 also illustrates an embodiment of a grip feature 512 in the form of a finger rest indentation in the pump's housing on a side thereof next to a side of the housing that attaches to a patient.

The user interface in this illustrated embodiment includes a plurality of lights including a first light 508 and a second light 510. The first light 508 is a single light extending circumferentially around an entire perimeter of the button 506. The second light 510 is a series of lights arranged in a line. The second light 510 includes four lights in this illustrated embodiment but can include another plural number of lights. The first and second lights 508, 510 are off (unilluminated) in FIG. 34.

The first and second lights 508, 510 are configured and used similar to the first and second lights 308, 310 of the pump 300 except that instead of each being located on a same side of the pump's body 502 like the first and second lights 308, 310 on the body 302 of the pump 300, the first light 508 is located on a same side of the body 502 as the button 506, and the second light 510 is located on a different side of the body 502 than the first light 508 and the button 506.

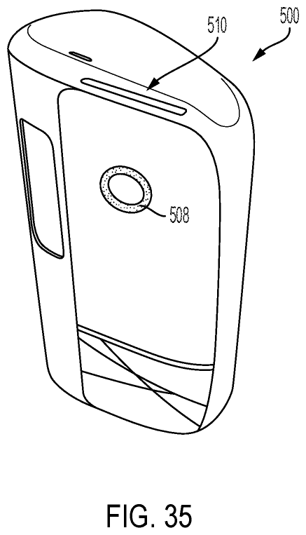

FIG. 35 shows the first light 508 on (illuminated) in a first color (e.g., green or other color) and the second light 510 off. The control circuitry can be configured to cause the first light 508 to be illuminated as in FIG. 35 in response to the manual removal of the backing or after the backing has been removed and the pump 500 adhered to a patient's skin, similar to that discussed above regarding the pump 300.

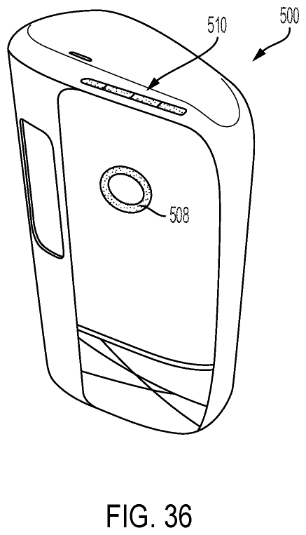

FIG. 36 shows the first light 508 on in the first color and the second light 510 on in the first color. The control circuitry can be configured to cause the second light 510 to be illuminated (either continuously or blinking) during priming. The first light 508 is on at this time (either continuously or blinking, as discussed above). The pump 500 can include a speaker (obscured in FIGS. 34-36) configured to provide an audio signal, e.g., one beep, a series of two beeps, a series of three beeps, a musical sequence, etc., as controlled by the control circuitry, when priming has been completed and prior to needle or cannula insertion into the patient. The audio signal can thereby indicate to the user that needle or cannula insertion is imminent.

Depression of the button 506 is configured to start drug delivery. In an exemplary embodiment, the first and second lights 508, 510 are configured to indicate to the user that pump 500 is ready to begin drug delivery by both of the first and second lights 508, 510 being illuminated in a continuous manner. The first light 508 is configured to indicate an orientation of the pump 500 to the user as discussed above, e.g., by being illuminated when the pump 500 is determined by the control circuitry to be is in an acceptable orientation for drug delivery based on data from the sensor monitoring pump 500 orientation. The second light 510 is configured to indicate drug delivery progress as discussed above and as discussed further below. FIG. 36 illustrates the pump 500 in a configuration in which the first and second lights 508, 510 indicate that the pump 500 is ready to begin drug delivery.

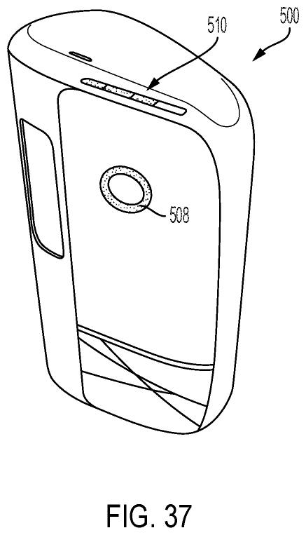

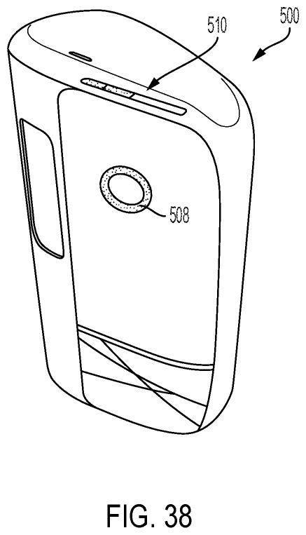

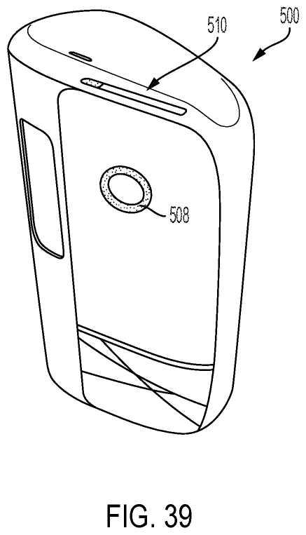

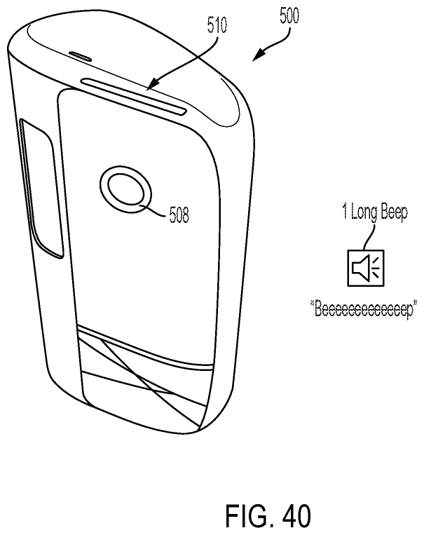

FIGS. 37-40 indicate a progress of drug delivery as indicated by the second light 510. The first light 508 remains in the illuminated state of FIG. 36 in FIGS. 37-40. The second light 510 acts as a countdown mechanism with lights in the series of lights being sequentially turned off as drug delivery occurs to indicate the drug delivery's progress. When drug delivery begins, e.g., when the button 506 is pressed, all four of the lights of the second light 510 are on. When drug delivery is about 25% complete, as determined by the control circuitry, an uppermost one of the light series of the second light 510 is turned off by the control circuitry such that three quarters of the light series is on and one quarter of the light series is off, as shown in FIG. 37. When drug delivery is about 50% complete, as determined by the control circuitry, a second one of the light series of the second light 510 is turned off by the control circuitry such that half of the light series is on and half the light series is off, as shown in FIG. 38. When drug delivery is about 75% complete, as determined by the control circuitry, a third uppermost one of the light series of the second light 510 is turned off by the control circuitry such that one quarter of the light series is on and three quarters of the light series is off, as shown in FIG. 39. When drug delivery is complete, as determined by the control circuitry, the fourth one of the light series of the second light 510 is turned off by the control circuitry such that all of the light series is off, as shown in FIG. 40. FIG. 40 also illustrates an audio signal being provided to also indicate that drug delivery is complete. The audio signal is one long beep in this illustrated embodiment, but another audio signal can be used to indicate end of drug delivery. In other embodiments with the second light 510 including a plural number of lights other than four, the lights will be sequentially turned off at different percentages of drug delivery completion.

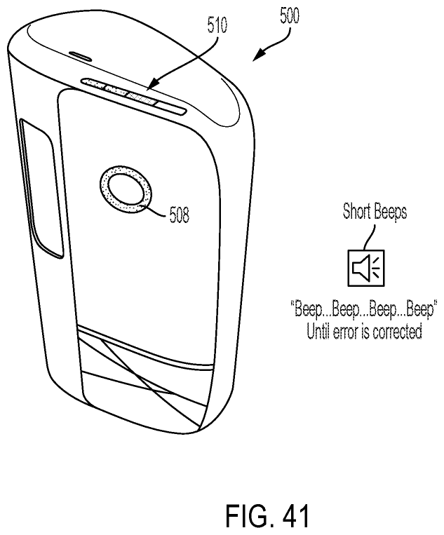

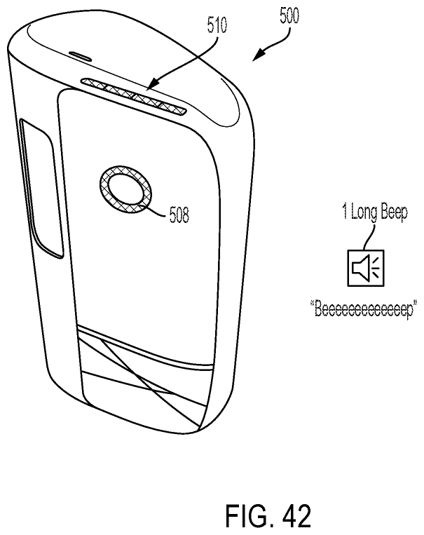

In the event that an error occurs during drug delivery, one or both of the first and second lights 508, 510 can be configured to indicate occurrence of the error, similar to that discussed above regarding the first and second lights 308, 310 of the pump 300. In an exemplary embodiment, the second light 510 is configured to indicate occurrence of an error during drug delivery by changing from the first color to a second color (e.g., red or other color). Which one of the series of lights in the second light 510 changes from the first color to the second color indicates a general timing of when the error occurred during drug delivery. For example, if an error occurs after about 75% of drug delivery is complete but before about 50% of drug delivery has been completed, e.g., when the second light 510 is illuminated as shown in FIG. 37, the second one of the light series, is changed from the first color to the second color by the control circuitry, as shown in FIG. 41. FIG. 41 also illustrates an audio signal being provided to also indicate that an error occurred. In this illustrated embodiment, the audio signal is series of short beeps that continue until the error is corrected, but another audio signal can be used to indicate error occurrence. If a fatal error is determined to have occurred, e.g., as determined by the control circuitry, that cannot be corrected, a so-called “fatal error,” each of the first and second lights 508, 510 can be configured to indicate occurrence of the fatal error. For example, as shown in FIG. 42, the control circuitry can be configured to cause each of the first and second lights 508, 510 to be in the second color in response to occurrence of a fatal error. FIG. 42 also illustrates an audio signal being provided to also indicate that a fatal error occurred. In this illustrated embodiment, the audio signal is continuous long beep, but another audio signal can be used to indicate fatal error occurrence.

FIG. 43 illustrates another embodiment of a pump 600 configured to be worn by a patient and to deliver a drug to the patient. The pump 600 of FIG. 43 is generally configured and used similar to the pump 400 of FIG. 21 except that the pump 600 of FIG. 43 includes a sticker 602 that covers the pump's button 604, which is surrounded by a light 606 similar to the light 408 surrounding the button 406 of the pump 400. The sticker 602 is configured to be manually removed by a user to expose the button 604, as shown in FIG. 44. The sticker 602 initially being positioned over the button 604 may help protect the button 604 before use of the pump 600, help protect the light 606 before use of the pump 606, and/or help ensure that the user knows the location of the button 604 and the light 606 since the sticker 602 being positioned over the button 604 prevents depression of the button 604. The sticker 602 is substantially rigid, e.g., made from a substantially rigid material, in at least a central portion thereof to help prevent depression of the button 604 until the sticker 602 is removed. A person skilled in the art will appreciate that an element may not be entirely rigid but nevertheless be considered to be substantially rigid due to any number of factors such as manufacturing tolerances and sensitivity of measurement equipment. The button 604 can be softer than the sticker 602, e.g., made from a softer material than the sticker 602, which may further indicate to the user that the pump 600 is ready for use with the sticker 602 removed. FIGS. 43 and 44 also illustrate an embodiment of a grip feature 608 in the form of a finger rest indentation in the pump's housing on a side thereof next to a side of the housing that attaches to a patient.

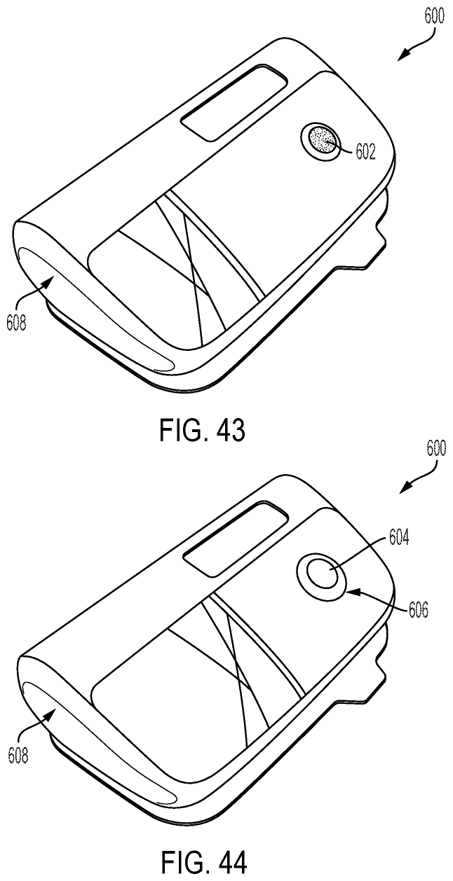

FIGS. 45 and 46 illustrate another embodiment of a pump 700 configured to be worn by a patient and to deliver a drug to the patient. The pump 700 of FIGS. 45 and 46 is generally configured and used similar to the pump 400 of FIG. 21 except that the pump 700 of FIGS. 45 and 46 has a differently shaped body 702 than the body 402 of the pump 400. FIGS. 45 and 46 each illustrate the pump's backing or label 704 on and the pump's light 706 off.

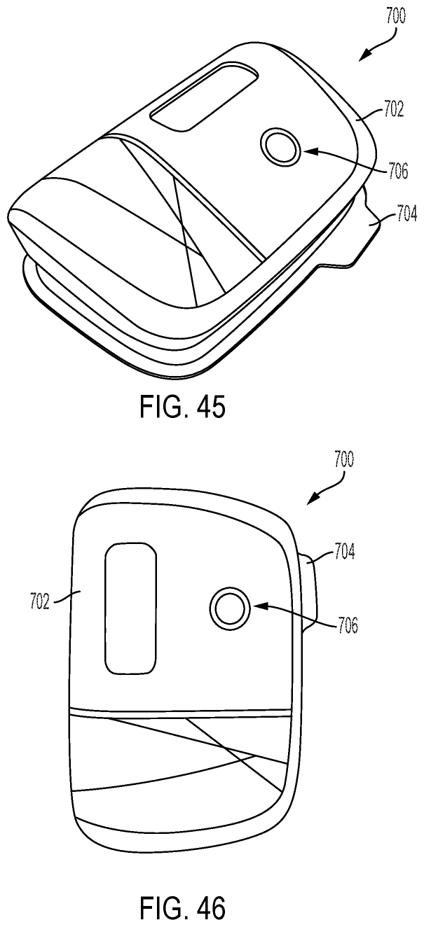

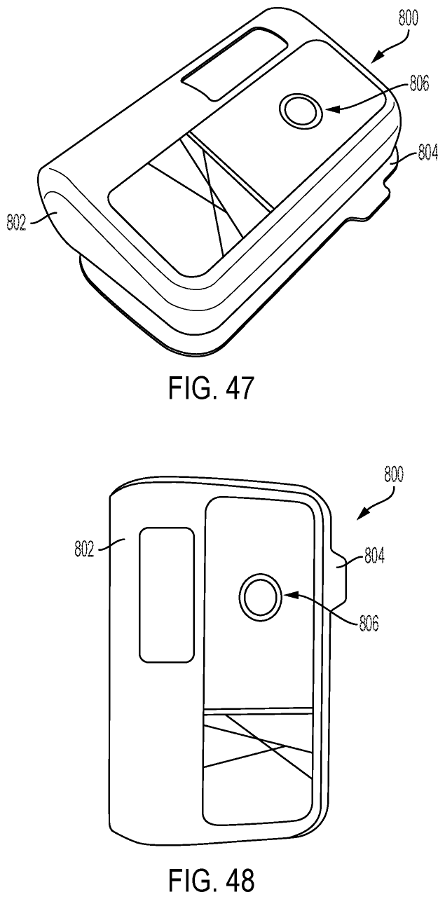

FIGS. 47 and 48 illustrate another embodiment of a pump 800 configured to be worn by a patient and to deliver a drug to the patient. The pump 800 of FIGS. 47 and 48 is generally configured and used similar to the pump 400 of FIG. 21 except that the pump 800 of FIGS. 47 and 48 has a differently shaped body 802 than the body 402 of the pump 400 (and than the pump 700 of FIGS. 45 and 46). FIGS. 47 and 48 each illustrate the pump's backing or label 804 on and the pump's light 806 off.

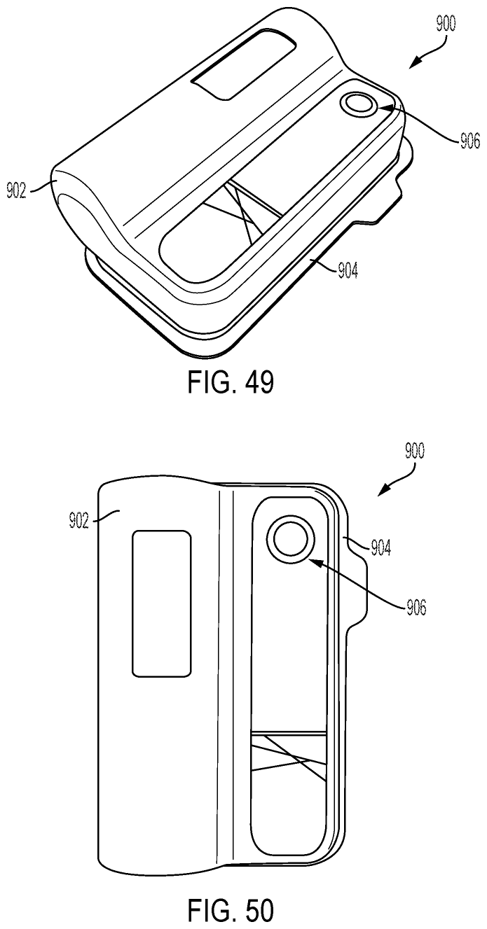

FIGS. 49 and 50 illustrate another embodiment of a pump 900 configured to be worn by a patient and to deliver a drug to the patient. The pump 900 of FIGS. 49 and 50 is generally configured and used similar to the pump 400 of FIG. 21 except that the pump 900 of FIGS. 49 and 50 has a differently shaped body 802 than the body 402 of the pump 400 (and than the pump 700 of FIGS. 45 and 46 and the pump 800 of FIGS. 47 and 48). FIGS. 49 and 50 each illustrate the pump's backing or label 904 on and the pump's light 906 off.

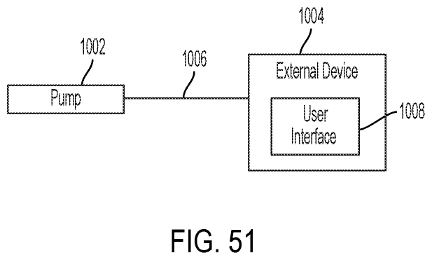

The pump 20 of FIG. 1, the pump 100 of FIGS. 8-10, the pump 200 of FIG. 11, the pump 300 of FIGS. 12-20, the pump 400 of FIGS. 21-33, the pump 500 of FIGS. 34-42, the pump 600 of FIGS. 43-44, the pump 700 of FIGS. 45-46, the pump 800 of FIGS. 47-48, and the pump 900 of FIGS. 49-50 each include a user interface on-board the pump that is configured to indicate an orientation of the pump to a user of the pump. In other embodiments, a user interface configured to indicate an orientation of a pump to a user of the pump can be off-board the pump but otherwise configured and used similar to the user interfaces discussed herein. FIG. 51 illustrates one embodiment of a system 1000 that includes a pump 1002 configured to be worn by a patient and to deliver a drug to the patient and an external device 1004 configured to communicate with the pump 1002 via a communication link 1006 (wired or wireless). The external device 1004 includes a user interface 1008 configured to indicate an orientation of the pump 1002 to a user of the pump 1002. Except for the user interface 1008 being off-board the pump 1002 instead of on-board the pump 1002, the pump 1002 of FIG. 51 is generally configured and used similar to any of the pump 20 of FIG. 1, the pump 100 of FIGS. 8-10, the pump 200 of FIG. 11, the pump 300 of FIGS. 12-20, the pump 400 of FIGS. 21-33, the pump 500 of FIGS. 34-42, the pump 600 of FIGS. 43-44, the pump 700 of FIGS. 45-46, the pump 800 of FIGS. 47-48, and the pump 900 of FIGS. 49-50.

In embodiments in which a user interface configured to indicate an orientation of a pump to a user of the pump is off-board the pump, the pump's control circuitry can include a communications interface (e.g., a wireless transceiver, etc.) configured to communicate, via wired or wireless connection, measured orientation data to an external device that is off-board of the pump and that includes the user interface. In an exemplary embodiment the communication interface is configured to communicate wirelessly using any of a number of wireless techniques, e.g., Wi-Fi, Near Field communication (NFC), Bluetooth, Bluetooth Low Energy (BLE), cellular communication, etc. The external device can be any of a variety of types of computer systems, such as a desktop computer, a workstation, a minicomputer, a laptop computer, a tablet computer, a personal digital assistant (PDA), a mobile phone, a smart watch, etc. The external device can be a personal device of the user and can be securely paired with the pump in any of a variety of ways, as will be appreciated by a person skilled in the art, to help ensure privacy and security.

The external device can have an application (also referred to herein as an “app”) installed thereon that controls the user interface that provides orientation information to the user. Data gathered by the pump's sensor can be communicated to the external device using the pump's communication interface and a corresponding communication interface of the external device that is configured to communicate with the pump's communication interface. The external device can be configured to provide orientation information as discussed herein to the user via the app, with the user interface controlled by the app including one or more of the varieties described herein, e.g., any one or more of light(s) on the external device, a vibrating mechanism, a speaker, and a display.

As discussed herein, one or more aspects or features of the subject matter described herein, for example components of the control circuitry, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

The present disclosure has been described above by way of example only within the context of the overall disclosure provided herein. It will be appreciated that modifications within the spirit and scope of the claims may be made without departing from the overall scope of the present disclosure.

1. A pump configured to deliver a liquid drug to a patient, comprising:
a reservoir configured to contain the liquid drug therein;
a pumping assembly configured to drive the liquid drug from the reservoir for delivery to the patient;
a sensor configured to measure an orientation of the pump;
a user interface defining a level configured to inform a user of an orientation of the pump; and
control circuitry configured to receive data from the sensor indicative of the measured orientation of the pump and to cause the user interface to provide an indication of the orientation of the pump to a user of the pump.

2. The pump of claim 1, wherein the user interface includes at least one of a light, a vibrating mechanism configured to vibrate, a speaker configured to provide an audio signal, a display configured to show information thereon, and a mechanical level.

3. The pump of claim 2, wherein the user interface includes at least the light.

4. The pump of claim 2, wherein the user interface includes at least the vibrating mechanism.

5. The pump of claim 2, wherein the user interface includes at least the speaker.

6. The pump of claim 2, wherein the user interface includes at least the display.

7. The pump of claim 2, wherein the user interface includes at least the mechanical level.

8. The pump of claim 1, wherein the control circuitry is configured to receive the data from the sensor as a series of real time pump orientation measurements and to cause the user interface to provide the indication of the orientation of the pump as a series of real time indications that each correspond to one of the real time pump orientation measurements.

9. The pump of claim 1, wherein the sensor includes at least one of an accelerometer, an inertial measurement unit (IMU), and a MARG (magnetic, angular rate, and gravity) sensor.

10. The pump of claim 1, further comprising a needle configured to be inserted into a patient;
wherein the pumping assembly is configured to drive the liquid from the reservoir and into the needle for delivery of the liquid drug into the patient.

11. The pump of claim 1, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

12. A method of using the pump of claim 1, comprising:
the control circuitry receiving the data from the sensor as a series of real time pump orientation measurements; and
the control circuitry causing the user interface to provide the indication of the orientation of the pump as a series of real time indications that each correspond to one of the real time pump orientation measurements.

13. The method of claim 12, further comprising activating the pumping assembly to move the liquid drug from the reservoir for delivery to the patient.

14. (canceled)

15. A pump configured to deliver a liquid drug to a patient, comprising:
a housing, the housing including a first side configured to be attached to skin of a patient at a recommended orientation relative to the patient;
a reservoir in the housing, the reservoir being configured to contain the liquid drug therein;
a pumping assembly in the housing, the pumping assembly being configured to drive the liquid drug from the reservoir for delivery to the patient;
a sensor in the housing, the sensor being configured to measure an orientation of the pump; and
a user interface located on a second side of the housing, the user interface being configured to be visible by the patient with the first side of the housing attached to the skin of the patient.

16. The pump of claim 15, further comprising control circuitry in the housing, the control circuitry being configured to receive data from the sensor indicative of the measured orientation of the pump and to cause the user interface to provide an indication of the orientation of the pump to the patient.

17. The pump of claim 16, wherein the control circuitry is configured to receive the data from the sensor as a series of real time pump orientation measurements and to cause the user interface to provide the indication of the orientation of the pump as a series of real time indications that each correspond to one of the real time pump orientation measurements.

18. The pump of claim 15, wherein the user interface includes at least one of a light, a display configured to show information thereon, and a mechanical level.

19. (canceled)

20. (canceled)

21. (canceled)

22. The pump of claim 15, wherein the sensor includes at least one of an accelerometer, an inertial measurement unit (IMU), and a MARG (magnetic, angular rate, and gravity) sensor.

23. The pump of claim 15, further comprising a needle configured to be inserted into a patient;
wherein the pumping assembly is configured to drive the liquid from the reservoir and into the needle for delivery of the liquid drug into the patient.

24. The pump of claim 15, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

25. A method of using the pump of claim 16, comprising:
the control circuitry receiving the data from the sensor as a series of real time pump orientation measurements; and
the control circuitry causing the user interface to provide the indication of the orientation of the pump as a series of real time indications that each correspond to one of the real time pump orientation measurements.

26. The method of claim 25, further comprising activating the pumping assembly to move the liquid drug from the reservoir for delivery to the patient.

27. (canceled)
